SEQUENCE LISTING (4 sequences)

SEQ ID NO: 1 - protein, 38 aa, synthetic construct
  SITE 1: note = 2-(3-cyano-5-fluorophenyl)-2-methylpropanoic acid modified Beta-alanine
  SITE 12: note = 2-Aminoisobutyric acid
  SITE 19: note = Residue attached to 2-(2-(2-aminoethoxy)ethoxy)acetic acid-2-(2-(2-aminoethoxy)ethoxy)acetic acid-gamma glu 19-carboxynonadecanoyl via the side chain
  SITE 38: note = Amidated residue
XEGTFTSDYSIXLDKIAQKAFVQWLIAGGPSSGAPPPS

SEQ ID NO: 2 - protein, 38 aa, synthetic construct; identical to SEQ ID NO: 1
  SITE 1: note = 2-(3-cyano-5-fluorophenyl)-2-methylpropanoic acid modified Beta-alanine
  SITE 4: note = Threonine attached to dimethyl pseudoproline via the side chain
  SITE 10: note = Serine attached to dimethyl pseudoproline via the side chain
  SITE 12: note = 2-Aminoisobutyric acid
  SITE 19: note = Residue attached to 2-(2-(2-aminoethoxy)ethoxy)acetic acid-2-(2-(2-aminoethoxy)ethoxy)acetic acid-gamma glu 19-carboxynonadecanoyl via the side chain

SEQ ID NO: 3 - protein, 38 aa, synthetic construct; identical to SEQ ID NO: 1
  SITE 1: note = Beta-alanine
  SITE 12: note = 2-Aminoisobutyric acid
  SITE 19: note = Residue attached to tert-butyl (2R,29S)-1,2-diamino -29-(tert-butoxycarbonyl)-1,8,17,26,31-pentaoxo-10,13,19,2 2-tetraoxa-7,16,25,30-tetraazapentacontan-50-oate via the side chain

SEQ ID NO: 4 - protein, 19 aa, synthetic construct
AFVQWLIAGGPSSGAPPPS

(19) United States
(12) Patent Application Publication
(10) Pub. No.: US 2026/0116848 A1
(43) Pub. Date: Apr. 30, 2026

(54) Synthesis of an Intermediate and Its Use for the Preparation of GLP-1R/GIPR Agonists

(71) Applicants: Carmot Therapeutics Inc., South San Francisco, CA (US); Hoffmann-La Roche Inc., Little Falls, NJ (US)

(72) Inventors: Shyam Krishnan, Novato, CA (US); Raphael Bigler, Basel (CH); Pascal Stephan Engl, Basel (CH); Carl Florian Falk, Basel (CH); Alec Fettes, Basel (CH); Michael Kammerer, Basel (CH); Christian Steffen Mössner, Basel (DE); Pascal Jarno Schmidt, Basel (DE); Helene Wolleb, Basel (CH); Martina Niess, Basel (CH); Philipp Stefan Weber, Basel (CH)

(73) Assignees: Carmot Therapeutics Inc., South San Francisco, CA (US); Hoffmann-La Roche Inc., Little Falls, NJ (US)

(21) Appl. No.: 19/287,728

(22) Filed: Jul. 31, 2025

Related U.S. Application Data
(60) Provisional application No. 63/678,026, filed on Jul. 31, 2024.

(51) Int. Cl. C07C 253/30; B01J 31/22; B01J 31/24; C07C 255/57; C07K 1/04; C07K 1/20

(52) U.S. Cl. CPC C07C 253/30; B01J 31/2282; B01J 31/2295; B01J 31/2409; C07C 255/57; C07K 1/042; C07K 1/20; B01J 2231/4205; B01J 2531/004; B01J 2531/0205; B01J 2531/824; B01J 2531/842

(57) ABSTRACT

The invention relates to processes for the synthesis of 2-(3-cyano-5-fluorophenyl)-2-methylpropanoic acid and its use as intermediate in the preparation of GLP-1R/GIPR agonists. The invention also relates to a process of preparing a GLP-1R/GIPR agonist.

### CROSS-REFERENCE TO RELATED APPLICATION

The present application claims priority from U.S. Application 63/678,026, filed Jul. 31, 2024. The contents of the priority application are incorporated by reference herein in its entirety.

### SEQUENCE LISTING

The instant application contains a Sequence Listing that has been submitted electronically in XML format and is hereby incorporated by reference in its entirety. Said XML copy, created on Jul. 30, 2025, is named 124921_US009_SL.xml and is 9,510 bytes in size.

### BACKGROUND OF THE INVENTION

The invention relates to novel processes for the synthesis of 2-(3-cyano-5-fluorophenyl)-2-methylpropanoic acid and its use as an intermediate in the preparation of GLP-1R/GIPR agonists. The invention also relates to an improved process of preparing GLP-1R/GIPR agonists, in particular a compound of formula (VII) (CT-388).

Glucagon-like peptide-1 (GLP-1) and gastric inhibitory polypeptide (GIP) are primary incretin hormones secreted from small intestinal L cells and K cells, respectively, on ingestion of glucose or nutrients to stimulate insulin secretion from pancreatic cells. The actions of GIP and GLP-1 are believed to be mediated by their receptors, the GIP receptor (GIPR) and the GLP-1 receptor (GLP-1R), respectively, which both belong to the G-protein coupled receptor family and are expressed in pancreatic cells, as well as in various tissues and organs. GLP-1R/GIPR agonists are compounds that mimic the action of the naturally occurring hormones GLP-1 and GIP. These hormones play a crucial role in regulating blood sugar levels by enhancing insulin secretion in response to meals, inhibiting glucagon release, and slowing gastric emptying. GLP-1R/GIPR agonists are thus useful medicaments in the treatment of type 2 diabetes mellitus to improve glycemic control. Additionally, they have been shown to promote weight loss, which can be beneficial for patients with obesity or those who are overweight. GLP-1R/GIPR agonists are effective in reducing HbAlc levels and have a favorable impact on cardiovascular outcomes in diabetic or overweight patients. Obesity is the most prevalent chronic disease worldwide and is associated with many other diseases.

There remains a high need for peptide mimetics that modulate the activity of GLP-1R and/or GIPR and, given the quantities of these peptide mimetics needed, for methods of synthesizing such chemical entities on a large scale.

### SUMMARY OF THE INVENTION

In one embodiment, this application relates to a process of preparing 2-(3-cyano-5-fluorophenyl)-2-methylpropanoic acid (formula (I)):

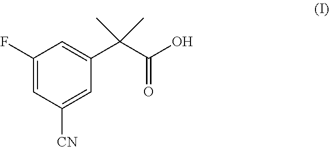

comprising a) reacting 3-bromo-5-fluorobenzonitrile (formula (II)):

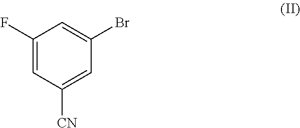

with an isobutyrate source in the presence of a palladium cross coupling catalyst to afford an ester of formula (III)

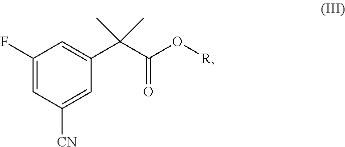

wherein R is C₁₋₄-alkyl, and b) hydrolyzing the ester of formula (III) to afford 2-(3-cyano-5-fluorophenyl)-2-methylpropanoic acid (formula (I)). In some embodiments, the palladium cross coupling catalyst is a Pd-trialkylphosphine complex catalyst. In some embodiments, step (a) comprises generating the palladium cross coupling catalyst in situ from a Pd precursor and a trialkylphosphine. The isobutyrate source may be a C₁₋₄-alkyl isobutyrate or a silyl ketene acetal. In some embodiments, the isobutyrate source is methyl isobutyrate or methyl trimethylsilyl dimethylketene acetal. In some embodiments, the isobutyrate source is methyl isobutyrate and the reacting takes place in the presence of a strong base. The strong base may be a lithium dialkylamide, which may be prepared in situ from an alkyl lithium compound and a dialkylamine. In some embodiments, the reacting in a non-polar organic solvent at a reaction temperature between-10° C. and 30° C. In some embodiments, the reacting of the palladium cross coupling catalyst with the silyl ketene acetal takes place in the presence of a Zn compound as co-activator. The silyl ketene acetal may be an alkyl trialkylsilyl dialkylketene acetal.

In some embodiments, step (a) takes place in a polar organic solvent at a reaction temperature between 40° C. and 100° C.

In some embodiments, step b) comprises hydrolyzing the ester of formula (III) with an alkali hydroxide. The alkali hydroxide may be lithium hydroxide. In some embodiments, step b) takes place in the presence of a non-polar organic solvent at a reaction temperature between 0° C. and 100° C. In some embodiments, step b) is carried out with lithium iodide.

Also provided is 2-(3-cyano-5-fluorophenyl)-2-methylpropanoic acid (formula (I)) produced according to any one of claims 1 to 15 for use in a method of preparing a compound of formula (VII), or a pharmaceutically acceptable salt thereof:

(VII)
[SEQ ID NO: 1]
X-β-Ala²-Glu³-Gly4-Thr⁵-Phe⁶-Thr⁷-Se^{r8}-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

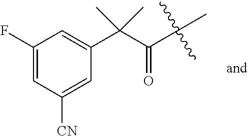 [SMILES: *C(=O)C(C)(C)c1cc(F)cc(C#N)c1]

AEEAc is 2-(2-(2-aminoethoxy) ethoxy) acetic acid.

Also provided is a process for preparing a compound of formula (VII), or a pharmaceutically acceptable salt thereof:

(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

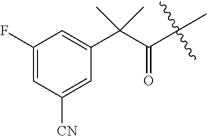 [SMILES: *C(=O)C(C)(C)c1cc(F)cc(C#N)c1]

and
AEEAc is 2-(2-(2-aminoethoxy) ethoxy) acetic acid, comprising: a) reacting 2-(3-cyano-5-fluorophenyl)-2-methylpropanoic acid (formula (I)) with the resin-bound peptide:

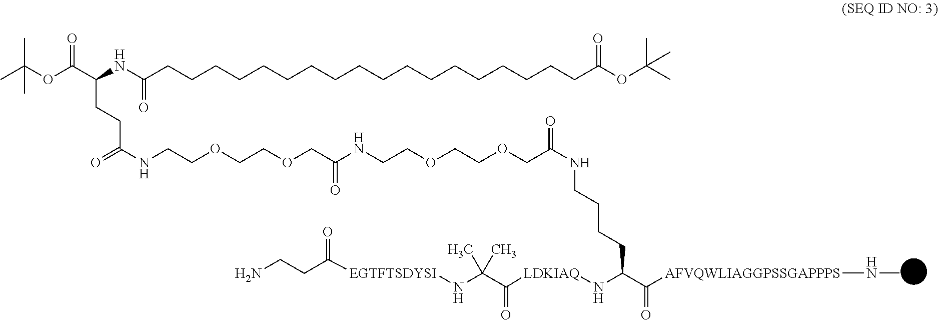

under amide bond-forming conditions.

In some embodiments, the process further comprises b) globally deprotecting and cleaving the compound of formula (VII) from the resin using an acid, c) precipitating the compound of formula (VII) or salt thereof, d) purifying the compound of formula (VII) or salt thereof, and e) isolating the compound of formula (VII) or salt thereof.

In some embodiments, the resin is an amide resin selected from the group consisting of Rink amide resin, Sieber amide resin, and Ramage amide resin (e.g., a Rink amide resin or Sieber amide resin).

In some embodiments, the solid phase synthesis comprises treating a resin-bound peptide with 10 to 30% piperidine in DMF, thereby deprotecting an Fmoc-protected amine of the resin-bound peptide.

In some embodiments, the solid phase synthesis comprises reacting ivDde-L-Lys(Fmoc)-OH or Mtt-L-Lys(Fmoc)-OH with the resin-bound peptide:

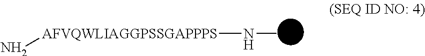

under amide bond-forming conditions.

In some embodiments, the solid phase synthesis comprises sequentially coupling each of the following to the resin or peptide intermediate bound thereto: (1) Fmoc-L-Ser(O′Bu)-OH, (2) Fmoc-L-Pro-OH, (3) Fmoc-L-Pro-OH, (4) Fmoc-L-Pro-OH, (5) Fmoc-L-Ala-OH, (6) Fmoc-Gly-OH, (7) Fmoc-L-Ser(O′Bu)-OH, (8) Fmoc-L-Ser(O′Bu)-OH, (9) Fmoc-L-Pro-OH, (10) Fmoc-Gly-Gly-OH, (11) Fmoc-L-Ala-OH, (12) Fmoc-L-Ile-OH, (13) Fmoc-L-Leu-OH, (14) Fmoc-L-Trp (Boc)-OH, (15) Fmoc-L-Gln(Trt)-OH, (16) Fmoc-L-Val-OH, (17) Fmoc-L-Phe-OH, (18) Fmoc-L-Ala-OH, (19) ivDde-L-Lys(Fmoc)-OH, (20) Fmoc-AEEA-OH, (21) Fmoc-AEEA-OH, (22) Fmoc-L-Glu-O′Bu, (23) 20-(tert-butoxy)-20-oxoicosanoic acid, followed by deprotection of ivDde with 3% v/v hydrazine monohydrate/DMF, (24) Fmoc-L-Gln(Trt)-OH, (25) Fmoc-L-Ala-OH, (26) Fmoc-L-Ile-OH, (27) Fmoc-L-Lys(Boc)-OH, (28) Fmoc-L-Asp(O′Bu)-OH, (29) Fmoc-L-Leu-OH, (30) Fmoc-Aib-OH, (31) Fmoc-L-Ile-OH, (32) Fmoc-L-Ser(O′Bu)-OH, (33) Fmoc-L-Tyr(O′Bu)-OH, (34) Fmoc-L-Asp(O′Bu)-OH, (35) Fmoc-L-Ser(O′Bu)-OH, (36) Fmoc-L-Thr(O′Bu)-OH, (37) Fmoc-L-Phe-OH, (38) Fmoc-L-Thr(O′Bu)-OH, (39) Fmoc-L-Gly-OH, (40) Fmoc-L-Glu (O′Bu)-OH, (41) Fmoc-β-Ala-OH, and (42) 2-(3-cyano-5-fluorophenyl)-2-methylpropanoic acid. In some embodiments, the solid phase synthesis comprises reacting a pseudoproline building block with a resin-bound peptide.

In some embodiments, the solid phase synthesis comprises sequentially coupling each of the following to the resin or peptide intermediate bound thereto: (1) Fmoc-L-Ser(O′Bu)-OH, (2) Fmoc-L-Pro-OH, (3) Fmoc-L-Pro-L-Pro-OH, (4) Fmoc-L-Ala-OH·H₂O, (5) Fmoc-Gly-OH, (6) Fmoc-L-Ser(O′Bu)-OH, (7) Fmoc-L-Ser(O′Bu)-OH, (8) Fmoc-L-Pro-OH·H₂O, (9) Fmoc-Gly-Gly-OH, (10) Fmoc-L-Ala-OH, (11) Fmoc-L-Ile-OH, (12) Fmoc-L-Leu-OH, (13) Fmoc-L-Trp (Boc)-OH, (14) Fmoc-L-Gln(Trt)-OH, (15) Fmoc-L-Val-OH, (16) Fmoc-L-Phe-OH, (17) Fmoc-L-Ala-OH, (18) ivDde-L-Lys(Fmoc)-OH, (19) Fmoc-AEEA-OH, (20) Fmoc-AEEA-OH, (21) Fmoc-L-Glu-O′Bu, (22) 20-(tert-butoxy)-20-oxoicosanoic acid, followed by deprotection of ivDde with 3% v/v hydrazine monohydrate/DMF, (23) Fmoc-L-Gln(Trt)-OH, (24) Fmoc-L-Ala-OH, (25) Fmoc-L-Ile-OH, (26) Fmoc-L-Lys(Boc)-OH, (27) Fmoc-L-Asp(O′Bu)-OH, (28) Fmoc-L-Leu-OH, (29) Fmoc-Aib-OH, (30) Fmoc-L-Ile-OH, (31) Fmoc-L-Tyr(′Bu)-L-Ser(Ψ(Me,Me)pro)-OH, (32) Fmoc-L-Asp(O′Bu)-OH, (33) Fmoc-L-Ser(O′Bu)-OH, (34) Fmoc-L-Thr(O′Bu)-OH, (35) Fmoc-L-Phe-OH, (36) Fmoc-Gly-L-Thr(Ψ(Me,Me)pro)-OH, (37) Fmoc-L-Glu (O′Bu)-OH, (38) Fmoc-β-Ala-OH, and (39) 2-(3-cyano-5-fluorophenyl)-2-methylpropanoic acid.

In some embodiments, the acid of step b) is trifluoroacetic acid (TFA). In some embodiments, step b) is carried out in a cleavage mixture comprising trifluoroacetic acid (TFA) and a scavenger. In some embodiments, the cleavage mixture of step b) comprises TFA, tri-isopropyl-silane (TIS), and water.

Step c) may be carried out using an anti-solvent, optionally wherein the anti-solvent is methyl t-butyl ether (TBME). Step d) may comprise preparative reversed phase HPLC purification, multicolumn countercurrent solvent gradient purification (MCSGP), and/or a desalting step. Step e) may comprise (i) a solvent/anti-solvent precipitating process, (ii) a spray drying process, (iii) freeze-drying, or a combination thereof. In some embodiments, the pharmaceutically acceptable salt of the compound of formula (VII) is an ammonium, potassium, sodium, acetate, chloride, or phosphate salt.

The invention relates to a novel process for the preparation of the intermediate 2-(3-cyano-5-fluorophenyl)-2-methylpropanoic acid of formula (I):

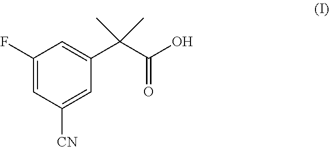

comprising
a) reacting 3-bromo-5-fluorobenzonitrile of formula II

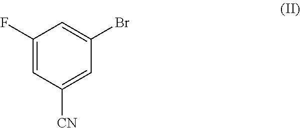

with an isobutyrate source in the presence of a palladium cross coupling catalyst to give the ester of formula III

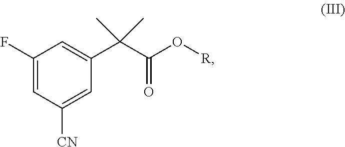

wherein R is C₁₋₄-alkyl, and
b) hydrolyzing the ester of formula III to give the 2-(3-cyano-5-fluorophenyl)-2-methylpropanoic acid of formula I.

In one aspect of the invention, the palladium cross coupling catalyst is a Pd-trialkylphosphine complex catalyst. In another aspect, the palladium cross coupling catalyst is generated in situ from a Pd-precursor and a trialkylphosphine.

In one aspect, hydrolyzing the ester of formula III is carried out with an alkali hydroxide.

The invention also relates to 2-(3-cyano-5-fluorophenyl)-2-methylpropanoic acid of the formula I produced according to the novel process described above for use in the preparation of a compound of formula VII (CT-388), or of a pharmaceutically acceptable salt thereof:

VII
[SEQ ID NO: 1]
X-β-Ala²-Glu³-Gly⁴-Thr⁵-Phe⁶-Thr⁷-Ser⁸-Asp⁹-
Tyr¹⁰-Ser¹¹-Ile¹²-Aib¹³-Leu¹⁴-Asp¹⁵-Lys¹⁶-Ile¹⁷-
Ala¹⁸-Gln¹⁹-Lys²⁰(AEEAc-AEEAc-γ-Glu-19-
carboxynonadecanoyl)-Ala²¹-Phe²²-Val²³-Gln²⁴-
Trp²⁵-Leu²⁶-Ile²⁷-Ala²⁸-Gly²⁹-Gly³⁰-Pro³¹-Ser³²-
Ser³³-Gly³⁴-Ala³⁵-Pro³⁶-Pro³⁷-Pro³⁸-Ser³⁹-NH₂

wherein X is

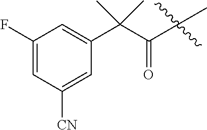 [SMILES: *C(=O)C(C)(C)c1cc(F)cc(C#N)c1]

and
AEEAc means 2-(2-(2-aminoethoxy) ethoxy) acetic acid.

Provided is also a process for the preparation of the compound of formula VII or of a pharmaceutically acceptable salt thereof:

VII
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

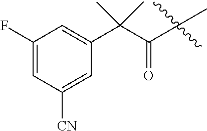 [SMILES: *C(=O)C(C)(C)c1cc(F)cc(C#N)c1]

and
AEEAc means 2-(2-(2-aminoethoxy) ethoxy) acetic acid,
wherein the process comprises the use of 2-(3-cyano-5-fluorophenyl)-2-methylpropanoic acid of formula I.

Also provided is a process for the preparation of the compound of formula VII or of a pharmaceutically acceptable salt thereof

VII
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

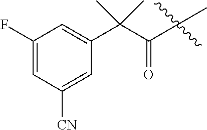 [SMILES: *C(=O)C(C)(C)c1cc(F)cc(C#N)c1]

and
AEEAc means for 2-(2-(2-Aminoethoxy) ethoxy) acetic acid, comprising the steps of
a) solid phase synthesis of the compound of formula VII on a resin, comprising in the last step adding the fragment of formula X by way of coupling a resin-bound peptide with 2-(3-cyano-5-fluorophenyl)-2-methylpropanoic acid,
b) cleavage of the compound of formula VII from the resin and global deprotection with an acid and
c) precipitation of the compound of formula VII,
d) purification, and
e) isolation of the compound of formula VII.

In one aspect, the solid phase synthesis of the peptide of formula VII is performed on an amide resin. In one aspect, the solid phase synthesis comprises Fmoc deprotection. In one aspect, the solid phase synthesis comprises pseudoproline building blocks. In one aspect, the acid used in the cleavage step is TFA. In one aspect, the purification comprises preparative reversed phase HPLC purification. In one aspect, the purification further comprises a modulation/desalting step.

In one aspect, the isolation of the compound of formula VII from the solution is performed by (i) a solvent/anti-solvent precipitation process, followed by filtration and drying, or (ii) a spray drying process, followed by an optional secondary drying step, or (iii) by freeze-drying, to obtain the compound of formula VII in high purity.

### BRIEF DESCRIPTION OF THE FIGURES

FIG. 1 is a diagram showing the solid-phase synthesis of a compound of formula (VII) using Sieber resin. The peptide shown has the sequence of SEQ ID NO: 1.

FIG. 2 is a diagram showing the solid-phase synthesis of a compound of formula (VII) using rink amide MBHA (methylbenzhydryl amine) resin. The peptide shown has the sequence of SEQ ID NO: 1.

### DETAILED DESCRIPTION OF THE INVENTION

This application is related, in part, to the compound 2-(3-cyano-5-fluorophenyl)-2-methylpropanoic acid (formula (I)) and its role as an intermediate in the synthesis of CT-388. Without wishing to be bound by theory, CT-388 is a dual GLP-1R/GIPR agonist that potently activates production of cyclic adenosine monophosphate (cAMP) but has no or minimal activity on the β-arrestin signaling pathways on either GLP-1R or GIPR. That is, CT-388 is fully biased towards cAMP activation, as opposed to being partially biased (i.e., with some β-arrestin activity) or unbiased (i.e., with full β-arrestin activity), on both GLP-1R and GIPR. β-arrestin activates kinase signaling pathways but also causes the GLP-1R and GIPR to be turned off and internalized. CT-388 does not cause internalization and consequently, desensitization of either GLP-1R or GIPR, and thus has enhanced signaling efficacy.

### I. Preparation of 2-(3-Cyano-5-Fluorophenyl)-2-Methylpropanoic Acid

In one embodiment, this application relates to a process for preparing the compound 2-(3-cyano-5-fluorophenyl)-2-methylpropanoic acid (formula (I)):

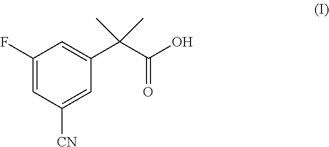

comprising
a) reacting 3-bromo-5-fluorobenzonitrile (formula (II):

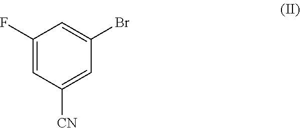

with an isobutyrate source in the presence of a palladium cross coupling catalyst to afford an ester of formula (III):

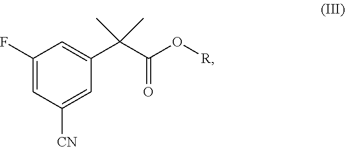

wherein R is C₁₋₄-alkyl, and
b) hydrolyzing the ester of formula III to afford the 2-(3-cyano-5-fluorophenyl)-2-methylpropanoic acid of formula (I).

Also provided herein are the compounds 2-(3-cyano-5-fluorophenyl)-2-methylpropanoic acid (formula (I)):

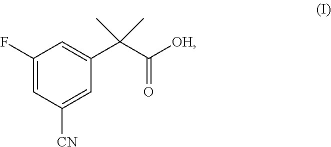

and
methyl 2-(3-cyano-5-fluorophenyl)-2-methylpropanoate (formula (IIIa)):

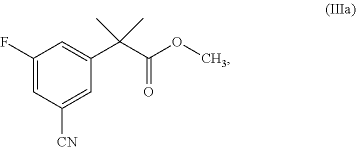 [SMILES: COC(=O)C(C)(C)c1cc(F)cc(C#N)c1]

as prepared by the process described above.

2-(3-cyano-5-fluorophenyl)-2-methylpropanoic acid (formula (I)) is a versatile intermediate, which, for instance, may provide certain functionality to compounds that modulate the glucagon-like peptide-1 receptor (GLP-1 receptor) as described in the International Patent Publication WO 2022/241287. Schemes A and B below illustrate the process as disclosed the synthesis of the structurally related 2-cyano-4-fluorophenyl and 2-fluoro-4-cyanophenyl derivatives, respectively.

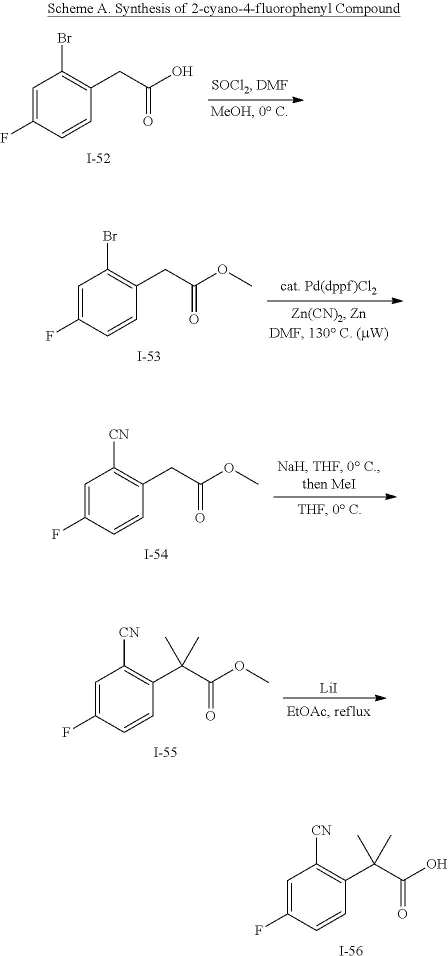

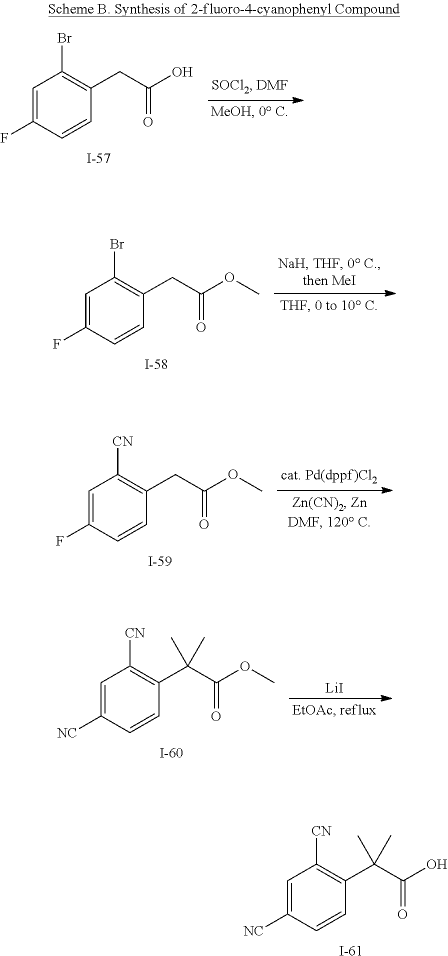

In an analogous manner, the 2-(3-cyano-5-fluorophenyl)-2-methylpropanoic acid of formula (I) may be prepared starting from 2-(3-bromo-5-fluorophenyl)-acetic acid and following one of the process routes shown in Scheme A or B. Each of these schemes requires four steps and the reaction conditions may not be suitable for a process on a larger scale.

This application describes a process for synthesizing 2-(3-cyano-5-fluorophenyl)-2-methylpropanoic acid (formula (I)) that has a number of steps and a set of reaction conditions that are suitable for a larger scale synthesis.

The following definitions are set forth to illustrate and define the meaning and scope of the various terms used to describe the invention herein.

The term “alkyl” as used herein denotes a monovalent linear or branched saturated hydrocarbon group of 1 to 12 carbon atoms. Typical examples include methyl, ethyl, n-propyl, i-propyl, n-butyl, i-butyl or t-butyl and pentyl, hexyl, heptyl, octyl, nonyl, decyl, undecyl or dodecyl and their isomers. The term “alkyl” as used herein also encompasses carbocycles. Examples for carbocycles include the monocyclic cyclopropyl, cyclobutyl, cyclopentyl or cyclohexyl or the polycyclic adamantyl.

The term “C₁₋₄-alkyl” denotes a monovalent linear or branched saturated hydrocarbon group of 1 to 4 carbon atoms. In some embodiments, the C₁₋₄-alkyl is methyl, ethyl, n-propyl, i-propyl, n-butyl, i-butyl or t-butyl. In some embodiments, “C₁₋₄-alkyl” denotes methyl or ethyl (e.g., methyl).

In one aspect, the invention relates to a novel process for preparing 2-(3-cyano-5-fluorophenyl)-2-methylpropanoic acid of formula (I):

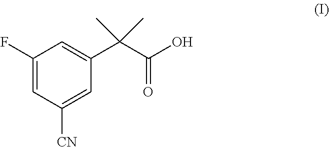

comprising
a) reacting 3-bromo-5-fluorobenzonitrile of formula (II)

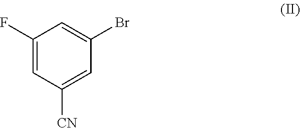

with an isobutyrate source in the presence of a palladium cross coupling catalyst to afford an ester of formula (III):

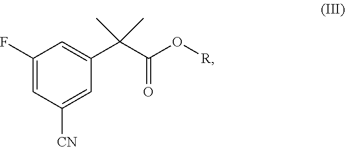

wherein is R is C₁₋₄-alkyl, and
b) hydrolyzing the ester of formula (III) to afford 2-(3-cyano-5-fluorophenyl)-2-methylpropanoic acid (formula (I)).

In a first embodiment, the first step comprises reacting 3-bromo-5-fluorobenzonitrile of formula (II):

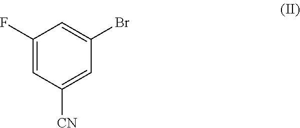

with an isooutyrate source in the presence of a palladium cross coupling catalyst to afford the ester of formula (III):

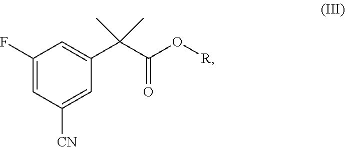

wherein R is C₁₋₄-alkyl.

The starting compound 3-bromo-5-fluorobenzonitrile (formula (II)) is commercially available.

In some embodiments, R is methyl, ethyl, propyl or n-butyl. In particular embodiments, R is methyl.

The cross coupling conditions may depend on the selection of the palladium (Pd) cross coupling catalyst and the isobutyrate source.

In one embodiment, the Pd-cross coupling catalyst is selected from a Pd-trialkylphosphine complex catalyst, such as a bis(tri-t-butylphosphine) palladium bromide (I), dimer, e.g. {[P(t-Bu)₃]PdBr}₂.

The isobutyrate source may be selected from a C₁₋₄-alkyl isobutyrate, such as methyl-, ethyl-, n-propyl, n-butyl, i-butyl or t-butyl isobutyrate. In some embodiments, the isobutyrate source is methyl isobutyrate.

In some embodiments, the reaction is carried out in the presence of a strong base. The strong base may be a lithium dialkylamide, which can be formed in situ from an organolithium compound and a dialkylamine.

Suitable organolithium compounds are alkyl lithium compounds such as methyl lithium, n-butyl lithium, t-butyl lithium, n-hexyllithium and phenyllithium. Suitable dialkylamines are dicyclohexylamine, diisopropylamine, cyclohexylisopropylamine or diethylamine. As an alternative, hexamethyldisilazane sodium salt (NaHMDS) may be employed without the use of an alkyl lithium compound. In some embodiments, the organolithium compound is n-butyl lithium.

The reaction may be performed in a suitable non-polar organic solvent, which may be a hydrocarbon (e.g., toluene) or an ether (e.g., cyclopentyl methyl ether, 2-methyltetrahydrofuran or anisole). In some embodiments, the non-polar organic solvent is toluene.

In some embodiments, the reaction temperature ranges between −10° C. and 30° C. In particular embodiments, the reaction temperature ranges between 0° C. and 20° C. In particular embodiments, the temperature ranges from 0-25° C. or 0-10° C.

The step of isolating the ester of formula (III), for example methyl 2-(3-cyano-5-fluorophenyl)-2-methylpropanoate (formula (IIIa)), from the reaction mixture may be carried out by procedures well known to the skilled in the art. For example, a suitable organic solvent such as methyl t-butyl ether, ethylacetate, n-propylacetate, i-propylacetate or 2-methyl tetrahydrofuran may be added.

The mixture may be adjusted to an acidic pH, the catalyst and inorganic salts may be filtered off, and/or the organic phase may be worked up by evaporation of the solvent and further purification, e.g. by chromatography. The resulting intermediate may be taken up in a suitable organic solvent such as tetrahydrofuran for making it readily available for the subsequent reaction step b).

In a second embodiment, the Pd-cross coupling catalyst is a Pd-trialkylphosphine complex catalyst generated in situ by bringing together a Pd-precursor (e.g., allylpalladium (II)-chloride dimer) and a suitable trialkyl phosphine (e.g., tris(1-adamantyl)phosphine). In some embodiments, a reaction carried out using a Pd-trialkylphosphine complex catalyst generated in situ may have the advantage that it can be carried out on a larger scale compared to a reaction carried out using a Pd-trialkylphosphine complex catalyst.

The isobutyrate source may be the same as selected for the first embodiment, i.e. can be a C₁₋₄-alkyl isobutyrate, such as methyl-, ethyl-, n-propyl, n-butyl, i-butyl or t-butyl isobutyrate. In some embodiments, the isobutyrate source is methyl isobutyrate.

In some embodiments, methyl isobutyrate is reacted with a Pd-cross coupling catalyst formed in situ by bringing together allylpalladium (II)-chloride dimer and tris(1-adamantyl)phosphine in the presence of a lithium dialkylamide base, in situ formed from n-butyllithium and dicyclohexylamine, in toluene at 0° C.

In some embodiments, the reaction conditions correspond to those mentioned in the first embodiment.

In a third embodiment, the reaction is a Pd-catalyzed enolate coupling with an enolate selected from a suitable silyl ketene acetal. In such embodiments, the Pd-cross coupling catalyst is a Pd-trialkylphosphine complex catalyst, which is generated in situ by bringing together a Pd-precursor such as tris(dibenzylidenacetone) dipalladium (0) and a suitable trialkyl phosphine or phosphonium salt, such as tri-t-butyl phosphoniumtetrafluoroborate.

In some embodiments, the silyl ketene acetal compound is a trialkylsilyl dialkylketene acetal compound (e.g., trimethylsilyl dimethylketene acetal).

In some embodiments, the reaction requires the presence of a Zn compound as additive. Suitable Zinc-compounds include, for instance, zinc fluoride or zinc chloride.

The reaction may be performed in a suitable organic solvent, such as a polar organic solvent like acetonitrile or N,N-dimethylformamide, or mixtures thereof. In some embodiments, the reaction is performed in a mixture of acetonitrile and N,N-dimethylformamide.

In some embodiments, the reaction temperature ranges between 40° C. and 100° C. (e.g., between 60° C. and 90° C.). In some embodiments, the reaction temperature is 80° C.

In some embodiments, the ester of formula (III) (e.g., methyl 2-(3-cyano-5-fluorophenyl)-2-methylpropanoate) is isolated from the reaction mixture by procedures well known to the skilled in the art. For example, the reaction mixture may be filtered to remove the catalyst, the organic phase may be concentrated, and the residue may be taken up in a suitable organic solvent such as ethyl acetate, the organic phase may be washed and finally the organic phase may be evaporated to obtain the desired intermediate. The resulting intermediate may be taken up in a suitable organic solvent (such as in tetrahydrofuran) for making it readily available for the subsequent reaction.

### Step (b)

The ester of formula (III) may be hydrolyzed to afford 2-(3-cyano-5-fluorophenyl)-2-methylpropanoic acid (formula (I)). Hydrolyzing the ester of formula (III) may be carried out with an alkali hydroxide, with the exception that acidic conditions may be required if R is t-butyl.

In some embodiments, a solution of the C₁₋₄-alkyl-2-(3-cyano-5-fluorophenyl)-2-methylpropanoate as obtained in step (a) may readily be subjected to step (b). The hydrolysis of the ester may be performed with an alkali hydroxide such as lithium, sodium, or potassium hydroxide in the presence of water. In some embodiments, the reaction is carried out with lithium hydroxide monohydrate in the presence of 1.5 equivalents of water.

An organic solvent, such as a non-polar solvent like dioxane, THF or 2-methyltetrahydrofuran (2-MeTHF) may be added. In some embodiments, the organic solvent is 1,4-dioxane. In some embodiments, the organic solvent is THF.

In some embodiments, the reaction temperature ranges between 0° C. and 100° C. (e.g., between 20° C. and 80° C.). In particular embodiments, the reaction temperature ranges between 0-25° C. In particular embodiments, the reaction temperature is 60° C.

In some embodiments, following the reacting step, a quenching step is carried out with a suitable acid such as aqueous hydrochloric acid or citric acid to adjust the reaction to an acidic pH. In some embodiments, the acid is 1 M aqueous HCl, and the quenching is carried out at 0° C.

The desired product may then be obtained by procedures known to the skilled in the art, for instance by extraction with a suitable organic solvent, such as with ethyl acetate or isopropyl acetate, removal of residual catalyst by treatment with activated carbon, and/or by removing or exchanging the solvent. In some embodiments, the process comprises removing residual Pd catalyst by treatment with silica-SH, optionally 20 weight percent. In some embodiments, the mixture is incubated at 60° C.

The step of purifying the product may comprise re-crystallization in a suitable organic solvent or via chromatography.

### II. Preparation of a Compound of Formula (VII)

Also provided herein is a process for preparing a compound of formula (VII), or a pharmaceutically acceptable salt thereof:

(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

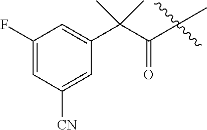 [SMILES: *C(=O)C(C)(C)c1cc(F)cc(C#N)c1]

and
AEEAc is 2-(2-(2-Aminoethoxy) ethoxy) acetic acid,
wherein the process comprises the use of 2-(3-cyano-5-fluorophenyl)-2-methylpropanoic acid (formula (I)).

The compound of formula (VII) [SEQ ID NO: 1] is referred to as “CT-388” and can also be shown in the format of formula (VIII):

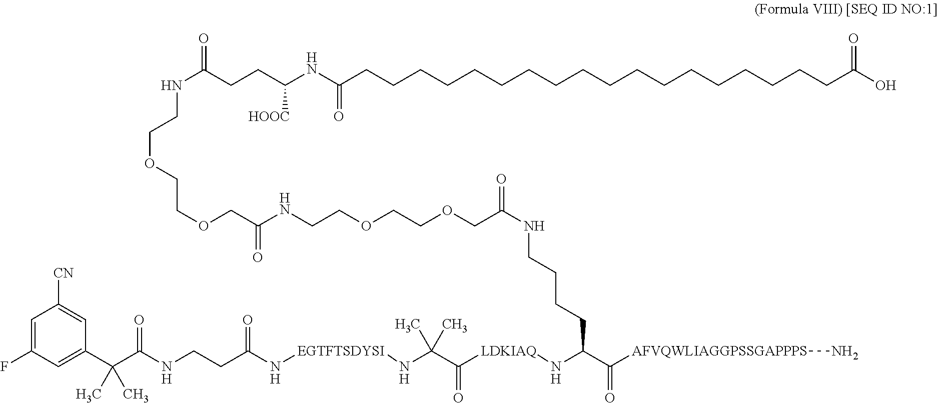

Formulas (VII) and (VIII) refer to the same compound (i.e., CT-388).

The compound 2-(3-cyano-5-fluorophenyl)-2-methylpropanoic acid (formula (I)) as produced according to the methods described herein may be useful in the process for the preparation of the compound of formula (VII), or of a pharmaceutically acceptable salt thereof:

VII
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂

wherein X is

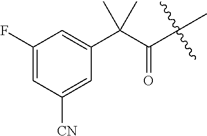 [SMILES: *C(=O)C(C)(C)c1cc(F)cc(C#N)c1]

and
AEEAc is 2-(2-(2-Aminoethoxy) ethoxy) acetic acid.

The compound of Formula (VII) is a GLP1-R/GIPR agonist. GLP1-R/GIPR agonists are peptides that may be synthesized via solid phase peptide synthesis (SPPS) methods.

SPPS is characterized by assembling the desired peptide chain on a solid support. The solid support may be a polymeric resin, for example a low cross-linked polystyrene bead, which is functionalized with reactive groups to enable covalent binding between the carboxyl group of the first amino acid of the nascent peptide chain and the resin through a linker. The most common polymeric solid support used is a resin composed of a 1-2% divinylbenzene-cross-linked polystyrene. Furthermore, a resin is composed of the polymeric solid support linked permanently to a linker (bifunctional spacer, or handle) that facilitates temporary anchoring of the first amino acid to the polymeric solid support. Depending on the type of linker, the C-terminus of the first amino acid is anchored to the solid support as an amide, ester, thioester, O-substituted oxime, or hydrazide. For instance, the Rink amide resin may comprise benzhydrylamine and the Sieber amide resin may comprise xanthenylamine. Alternatively, Ramage amide resin (tricyclic amide linker resin) may be used. Each of these resins forms an amide linkage with the first amino acid. After being cleaved from one of these solid supports, the peptide comprises a C-terminal amide group.

Each amino acid to be coupled to the peptide chain N-terminus must bear an appropriate protective group on its α-amino group and potentially on its side chain. The base-labile Fluorenylmethyloxycarbonyl (Fmoc) group may be used to protect the α-amino group of the incoming amino acid. The repeated cycles involve alternate deprotection and coupling reactions.

In some embodiments, the coupling reactions require activation of the carboxylic acid moiety with coupling agents or activators such as N,N′-diisopropylcarbodiimide (DIC) to support an effective amide bond formation. Conditions of a coupling reaction may determine the acylation rate, as well as the extent of side reactions, such as racemization.

Racemization may be circumvented with “racemization suppressing” additives such as 1-hydroxy-benzotriazole (HOBt), 1-hydroxy-7-aza-benzotriazole (HOAt), or ethyl cyano(hydroxyimino)acetate (OxymaPure). OxymaPure has been developed as an efficient additive for carbodiimide coupling. In particular embodiments, the amide bond forming conditions comprise use of DIC and oxymapure.

The use of α-amino group and side chain protecting groups is essential during peptide synthesis to avoid undesirable side reactions, such as self-coupling of the activated amino acid. Two principle protecting group schemes are typically used in solid phase peptide synthesis. The t-butyloxycarbonyl (Boc)/benzyl (Bzl) strategy utilizes TFA-labile N-terminal Boc protection alongside side chain protection that is removed simultaneously with cleavage of the peptide from the solid support using anhydrous hydrogen fluoride (HF) during the final cleavage step. The fluorenylmethyloxycarbonyl (Fmoc)/t-butyl (tBu) strategy uses base-labile Fmoc N-terminal protection whereas the side chain protection and the resin linkage are acid-labile, and the final acidic cleavage is carried out with an acid such as TFA.

At the end of the synthesis, the crude peptide may be cleaved from the solid support while simultaneously removing all protecting groups, in some embodiments using an acidic reagent such as trifluoroacetic acid, and optionally purified.

While the reaction conditions of the SPPS must be adapted for every peptide, the general SPPS method is well known in the art and is described for instance in detail in W. C. Chan and P. D. White, Fmoc Solid Phase Peptide Synthesis: A Practical Approach, Oxford University Press Inc., 1999.

Provided herein is thus a process for the preparation of the compound of formula (VII) (CT-388), or of a pharmaceutically acceptable salt thereof:

VII
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴⁻Ala³⁵-Pro³⁶-Pro³⁷-Pro³⁸-Ser³⁹-NH₂

wherein X is

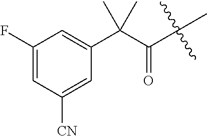 [SMILES: *C(=O)C(C)(C)c1cc(F)cc(C#N)c1]

and
AEEAc is 2-(2-(2-Aminoethoxy) ethoxy) acetic acid, comprising the steps of
a) solid phase synthesizing the compound of formula (VII) on a resin, comprising in the last step reacting 2-(3-cyano-5-fluorophenyl)-2-methylpropanoic acid carboxylic acid (formula (I)) with the resin-bound peptide,
b) cleaving the compound of formula (VII) from the resin and globally deprotecting the peptide with an acid (e.g., TFA or TFA in combination with 1,4-dithiothreitol (DTT)),
c) precipitating the compound of formula (VII) or pharmaceutically acceptable salt thereof,
d) purifying the compound of formula (VII) or pharmaceutically acceptable salt thereof, and/or
e) isolating the compound of formula (VII) or pharmaceutically acceptable salt thereof.

### Step (a): Solid Phase Synthesis

In one embodiment, the solid phase synthesis of the compound of formula (VII) is performed on an amide resin. In one aspect, the amide resin is selected from the group consisting of Rink amide resin, Sieber amide resin and Ramage amide resin. In a particular embodiment, the amide resin is a Rink amide resin or Sieber amide resin.

In one aspect, the solid phase synthesis comprises deprotecting an Fmoc group.

In one particular aspect, the solid phase synthesis of the compound of formula (VII) comprises sequentially coupling the following compounds to the nascent resin-bound peptide:
(1) Fmoc-L-Ser(O′Bu)-OH,
(2) Fmoc-L-Pro-OH,
(3) Fmoc-L-Pro-OH,
(4) Fmoc-L-Pro-OH,
(5) Fmoc-L-Ala-OH,
(6) Fmoc-Gly-OH,
(7) Fmoc-L-Ser(O′Bu)-OH,
(8) Fmoc-L-Ser(O′Bu)-OH,
(9) Fmoc-L-Pro-OH,
(10) Fmoc-Gly-Gly-OH,
(11) Fmoc-L-Ala-OH,
(12) Fmoc-L-Ile-OH,
(13) Fmoc-L-Leu-OH,
(14) Fmoc-L-Trp (Boc)-OH,
(15) Fmoc-L-Gln(Trt)-OH,
(16) Fmoc-L-Val-OH,
(17) Fmoc-L-Phe-OH,
(18) Fmoc-L-Ala-OH,
(19) ivDde-L-Lys(Fmoc)-OH,
(20) Fmoc-AEEA-OH,
(21) Fmoc-AEEA-OH,
(22) Fmoc-L-Glu-O′Bu,
(23) 20-(tert-butoxy)-20-oxoicosanoic acid, followed by deprotection of ivDde with hydrazine monohydrate/DMF,
(24) Fmoc-L-Gln(Trt)-OH,
(25) Fmoc-L-Ala-OH,
(26) Fmoc-L-Ile-OH,
(27) Fmoc-L-Lys(Boc)-OH,
(28) Fmoc-L-Asp(O′Bu)-OH,
(29) Fmoc-L-Leu-OH,
(30) Fmoc-Aib-OH,
(31) Fmoc-L-Ile-OH,
(32) Fmoc-L-Ser(O′Bu)-OH,
(33) Fmoc-L-Tyr(O′Bu)-OH,
(34) Fmoc-L-Asp(O′Bu)-OH,
(35) Fmoc-L-Ser(O′Bu)-OH,
(36) Fmoc-L-Thr(O′Bu)-OH,
(37) Fmoc-L-Phe-OH,
(38) Fmoc-L-Thr(O′Bu)-OH,
(39) Fmoc-Gly-OH,
(40) Fmoc-L-Glu (O′Bu)-OH, and
(41) Fmoc-β-Ala-OH, and
(42) 2-(3-cyano-5-fluorophenyl)-2-methylpropanoic acid.

In some embodiments, the solid phase synthesis comprises the steps of coupling ivDde-L-Lys(Fmoc)-OH, a lysine building block comprising the orthogonal protecting group 1-(4,4-dimethyl-2,6-dioxocyclohex-1-ylidene)-3-methylbutyl (ivDde) that can be cleaved under different conditions than Fmoc, followed by stepwise coupling with Fmoc-AEEA-OH (two steps), Fmoc-L-Glu-O′Bu and 20-(tert-butoxy)-20-oxoicosanoic acid, and then deprotection of the α-amino protecting group ivDde with hydrazine monohydrate and DMF.

As used herein, the abbreviation AEEAc is used to represent the compound 2-(2-(2-aminoethoxy) ethoxy) acetic acid or, where appropriate, a monovalent or divalent fragment thereof. A person having ordinary skill in the art would recognize that, when being part of a larger molecule, “AEEAc” has the following bivalent structure:

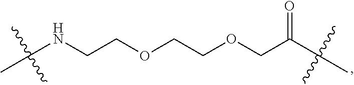 [SMILES: *NCCOCCOCC(*)=O]

wherein each [symbol] represents a point of attachment to the remainder of the molecule.

In some embodiments, the solid phase synthesis of the compound of formula (VII) comprises reacting a pseudoproline building block with the nascent resin-bound peptide.

In particular embodiments, the orthogonal protecting group Mtt (4-methyltrityl) may be used. In such embodiments, the solid phase synthesis comprises the steps of coupling Mtt-L-Lys(Fmoc)-OH, followed by stepwise coupling with Fmoc-AEEA-OH (two steps), Fmoc-L-Glu-O′Bu and 20-(tert-butoxy)-20-oxoicosanoic acid, and then selective removal of the α-amino protecting group Mtt under mild acidic conditions. The mild acidic conditions may be, for example, dilute TFA in dichloromethane, with hexafluoroisopropanol (HFIP), or with the mixture of hexafluoroisopropanol (HFIP) and trisopropylsilane (TIPS) in an organic solvent, e.g., dichloromethane.

Pseudoproline building blocks are artificially created dipeptides that consist of serine- or threonine-derived oxazolidines and cysteine-derived thiazolidines. The name pseudoproline is based on the structural similarity with the cyclic amino acid proline. The presence of Y Pro within a peptide sequence results in the disruption of β-sheet peptide structures that are considered as a source of intermolecular aggregation. Thus, compared to the standard peptide, the pseudoproline dipeptides prevent or limit the formation of aggregates and thus allow the activated amino acid derivative to better assemble with the N-terminus of the growing peptide chain. Incorporating pseudoproline dipeptides into the peptide sequence may enhance the coupling efficacy, and in turn, improve the purity and yield of the peptide. When treated with trifluoroacetic acid (TFA), the pseudoproline peptides are converted into serine, threonine or cysteine. For conciseness, when drawing nascent resin-bound peptides (i.e., prior to treatment with TFA), protecting groups (e.g., OtBu on Ser or Trt on Gln) and pseudoproline residues (e.g., Ser ((Me,Me)pro)) are omitted. A person having ordinary skill in the art would recognize that these groups remain present on the peptide until it has been treated with acid, even if they are not explicitly drawn in these nascent resin-bound structures.

In some embodiments, the solid phase synthesis of the compound of formula (VII) comprises sequentially coupling the following to a nascent resin-bound peptide:
(1) Fmoc-L-Ser(O′Bu)-OH,
(2) Fmoc-L-Pro-OH·H₂O,
(3) Fmoc-L-Pro-L-Pro-OH,
(4) Fmoc-L-Ala-OH,
(5) Fmoc-Gly-OH,
(6) Fmoc-L-Ser(O′Bu)-OH,
(7) Fmoc-L-Ser(O′Bu)-OH,
(8) Fmoc-L-Pro-OH,
(9) Fmoc-Gly-Gly-OH,
(10) Fmoc-L-Ala-OH,
(11) Fmoc-L-Ile-OH,
(12) Fmoc-L-Leu-OH,
(13) Fmoc-L-Trp (Boc)-OH,
(14) Fmoc-L-Gln(Trt)-OH,
(15) Fmoc-L-Val-OH,
(16) Fmoc-L-Phe-OH,
(17) Fmoc-L-Ala-OH,
(18) ivDde-L-Lys(Fmoc)-OH,
(19) Fmoc-AEEA-OH,
(20) Fmoc-AEEA-OH,
(21) Fmoc-L-Glu-O′Bu,
(22) 20-(tert-butoxy)-20-oxoicosanoic acid, followed by deprotection of ivDde with hydrazine monohydrate/DMF,
(23) Fmoc-L-Gln(Trt)-OH,
(24) Fmoc-L-Ala-OH,
(25) Fmoc-L-Ile-OH,
(26) Fmoc-L-Lys(Boc)-OH,
(27) Fmoc-L-Asp(O′Bu)-OH,
(28) Fmoc-L-Leu-OH,
(29) Fmoc-Aib-OH,
(30) Fmoc-L-Ile-OH,
(31) Fmoc-L-Tyr(′Bu)-L-Ser(ΨMe,Me)pro)-OH,
(32) Fmoc-L-Asp(O′Bu)-OH,
(33) Fmoc-L-Ser(O′Bu)-OH,
(34) Fmoc-L-Thr(O′Bu)-OH,
(35) Fmoc-L-Phe-OH,
(36) Fmoc-Gly-L-Thr(Ψ(Me,Me)pro)-OH,
(37) Fmoc-L-Glu (O′Bu)-OH,
(38) Fmoc-β-Ala-OH, and
(39) 2-(3-cyano-5-fluorophenyl)-2-methylpropanoic acid.

### Step (b): Cleaving and Deprotecting

Once the synthesis on solid phase is complete, cleaving the compound of formula (VII) from the resin and globally deprotecting with an acid follows.

Cleaving the compound of formula (VII) from resin along with the concomitant cleavage of protecting groups may be performed by commonly used cleavage mixtures, for example cleavage mixtures comprising trifluoroacetic acid (TFA) in combination with scavengers. Scavengers are substances that prevent either side reactions mediated by the released protecting groups or the side chains themselves. Common cleavage cocktail mixtures comprise TFA, tri-isopropyl-silane (TIS), and H₂O (for instance 90:5:5 v/v/v TFA/TIS/H₂O). Alternatively, cocktail mixtures with TFA and/or thiol based scavengers, such as Ethan-1,2-dithiol (EDT), 1,4-dithiothreitol (DTT), 1,4-dithioerythritol (DTE) or 2,2′-(ethylenedioxy) diethanethiol (DODT), or mixtures of TFA, Tis, H₂O and thiol based scavengers may be used.

In one particular embodiment, the step of cleaving the compound of Formula (VII) from the resin and globally deprotecting is carried out with a cocktail mixture of TFA and 1,4-dithiothreitol (DTT). In some embodiments, the cleaving step is carried out using a mixture of TFA, water, and TIS.

### Step (c): Precipitating a Compound of Formula (VII)

The synthesis and purification of a compound of formula (VII), or a pharmaceutically acceptable salt thereof, may comprise a precipitating step, wherein the compound or salt is precipitated as a crude peptide from solution using an anti-solvent, such as methyl t-butyl ether (TBME), diethyl ether, or diisopropylether. In particular embodiments, the anti-solvent is TBME.

### Step (d) Purifying a Compound of Formula (VII)

The crude (e.g., precipitated) peptide or salt thereof may be dissolved in an appropriate solvent or solvent mixture, e.g., a mixture of organic solvent and water. The organic solvent may be chosen from acetonitrile (MeCN), ethanol, isopropanol, methanol, butanol and/or tetrahydrofuran. The solution may be heated (to, e.g., 40° C.) to facilitate decarboxylation. The solution may be adjusted to neutral to basic pH in the range of 7.0 to 10, optionally in the pH range from 7.0 to 8.5, further optionally to pH 8.0 by adding an aqueous inorganic base, preferably chosen from the group consisting of acetate, carbonate, citrate, formate, hydroxide, and phosphate in their ammonium, potassium or sodium form. In particular embodiments, the aqueous inorganic base is ammonium hydroxide. In some embodiments, the solution is stirred at room temperature and then diluted to the final concentration for purification.

The pre-treated solution may be purified by preparative reversed-phase HPLC. The stationary phase may be selected from C4, C6, C8, C12, C18, C20, di-C4, cyano, or phenyl-modified silica resins or polymeric resins with pore sizes between 1 to 100 nm and particle sizes between 1 to 100 μm. In a particular embodiment, the stationary phase is a C8 or C18 modified silica resin with a pore size between 10 to 30 nm and a particle size of 10 μm. In some embodiments, the stationary phase is Kromasil 100-10-C18 or Daisopak 100-10-C8.

The mobile phase may be a mixture of organic solvent and water. The organic solvent may be chosen from acetonitrile, butanol, ethanol, isopropanol, methanol, and/or tetrahydrofuran. In particular embodiments, the organic solvent is acetonitrile. Buffer additives may be selected from acetate, carbonate, chloride, citrate, dihydrogen phosphate, formate, hydrogencarbonate, monohydrogen phosphate, phosphate, or trifluoroacetate in their free acid, ammonium, potassium or sodium form. In particular embodiments, the buffer additives are selected from the group consisting of ammonium acetate, sodium acetate, ammonium dihydrogen phosphate, sodium dihydrogen phosphate, and trifluoroacetic acid.

The purifying step may be run on an automated pump skid. The automatic pump skid may control the composition of the buffer mixture and enable fractionation by setting a defined UV trigger and collecting fractions by time or volume. The product may be eluted using a linear gradient, step gradient, or combination of linear and step gradient with increasing or partially constant organic content. The purification may comprise multiple sequential chromatographic steps, for example two to three chromatographic steps. In some embodiments, the peptide is eluted from the column and the effluent is fractionated and assayed for purity using reversed phase HPLC-UV analysis. The suitable fractions may be pooled after analysis based on their purity. Fractions not meeting the pooling criteria may be combined for recycle injections. Recycle injections are processed and pooled using the primary injection criteria. The whole purification process including the recycling of impure fractions can be fully automated using a multicolumn countercurrent solvent gradient purification (MCSGP) approach using absolute UV, relative UV, time-based criteria, or combinations thereof for recycling the weak adsorbing overlap and strong adsorbing overlap regions. Effluent from weak and strong adsorbing overlap regions are inline diluted with low organic content buffer before loading on the next column.

The purification process can be performed using one or two dimensions. In some embodiments, the purification process is performed using one dimension.

In a particular embodiment, the precipitated crude peptide (comprising the compound of formula (VII)) is solubilized in water/MeCN mixtures and then purified by preparative HPLC using water/MeCN as eluents that may be modified by TFA, ammonium bicarbonate, or ammonium acetate.

In some embodiments, the purifying step comprises use of multicolumn countercurrent solvent gradient purification (MCSGP). The MCSGP step may comprise the use of two columns, optionally packed with stationary phase Daisopak 100-10-C8.

In addition, the purifying step may further comprise a modulating step to remove unwanted inorganic salts and/or the solvent may be exchanged. In some embodiments, the modulating step is a desalting step, and is performed via preparative reversed phase HPLC purification at ambient temperature by applying isocratic gradients varying in organic modifier content of an aqueous eluent system to remove inorganic salts. The peptide may be eluted from the column and the effluent may be fractionated and assayed for purity using reversed phase HPLC-UV analysis. Suitable fractions that qualify by purity and impurity content may be pooled. To achieve an acceptable concentration for the following isolation step, the solution may be concentrated by e.g., distillation.

In some embodiments, the desalting step is carried out on a pre-equilibrated Kromasil 100-10-C18 packed column. In some embodiments, a tangential flow filtration (TFF) step is used to desalting and/or change the solvent.

### Step (e): Isolating a Compound of Formula (VII)

In the isolating step, the compound of formula (VII) or salt thereof may be isolated from the solution obtained in the purifying and/or modulating step either by (i) a solvent/anti-solvent precipitation process, followed by filtration and drying, (ii) a spray drying process, followed by an optional secondary drying step, or (iii) freeze-drying.

In some embodiments, the isolating step comprises a solvent/anti-solvent precipitating process, followed by filtering and drying. In such embodiments, precipitation of the peptide is achieved by adding the anti-solvent to the peptide solution or vice versa under sufficient stirring. The reaction mixture may be allowed to sit and the compound or salt may be isolated by filtration. The wet filter cake may be dried e.g., by usage of a nitrogen stream or a filter dryer. In some embodiments, the precipitating step comprises use of TBME.

In some embodiments, the isolating step comprises a spray drying process, followed by an optional secondary drying step. In some embodiments, the peptide powder is generated in a spray drying process by atomization of the peptide solution into droplets e.g., by applying pressure and bringing these droplets into contact with the heated nitrogen. The generated powder may be collected in e.g., a cyclone or a filter bag. In case the limits for residual solvent or water are not met, the powder may be dried in a secondary drying step using, e.g., an agitated contact dryer or a vacuum tray oven. In some embodiments, the spray drying step is carried out on a Büchi S-300 laboratory spray dryer with an inert loop S-395. In some embodiments, the spray drying is carried out in a 70% (w/w) aqueous ethanol solution. In some embodiments, the spray drying is carried out in a 70% (w/w) aqueous 2-propanol solution.

In some embodiments, the isolating step comprises a freeze-drying step. In some embodiments, the peptide is isolated by freezing the peptide solution after modulation and removal of solvent and water by sublimation and secondary drying at defined temperatures and holding times under reduced pressure. If needed, the initial modulation solution may be diluted with water to reduce the content of the organic solvent.

The term “pharmaceutically acceptable salt” refers to those salts which retain the biological effectiveness and properties of the free bases or free acids, which are not biologically or otherwise undesirable. The salts may be formed with inorganic acids such as hydrochloric acid, hydrobromic acid, sulfuric acid, nitric acid, phosphoric acid and the like. In some embodiments, the salt is a hydrochloric acid salt. In some embodiments, the salt is an organic acid such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, or N-acetylcysteine salt, and the like.

In some embodiments, a pharmaceutically acceptable salt may be prepared by adding an inorganic base or an organic base to the free acid. Salts derived from an inorganic base include, but are not limited to, the sodium, potassium, lithium, ammonium, calcium, magnesium salts of a compound and the like. Salts derived from organic bases include but are not limited to salts of primary, secondary, and tertiary amines, substituted amines, including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins, and the like.

In some embodiments, a pharmaceutically acceptable salt of the compound of formula (VII) is isolated. In some embodiments, compound forms a pharmaceutically acceptable salt with a cationic counterion (e.g., ammonium, potassium, or sodium). In some embodiments, the compound forms a pharmaceutically acceptable salt with an anionic counterion (e.g., acetate, chloride, or phosphate).

### III. Exemplary Embodiments

Non-limiting, exemplary embodiments are provided below to further illustrate the present disclosure.
1. A process of preparing 2-(3-cyano-5-fluorophenyl)-2-methylpropanoic acid (formula (I)):

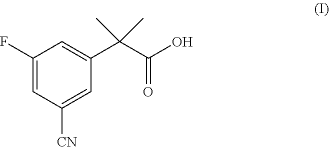

comprising
a) reacting 3-bromo-5-fluorobenzonitrile (formula (II)):

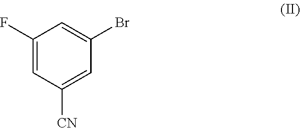

with an isobutyrate source in the presence of a palladium cross coupling catalyst to afford an ester of formula (III)

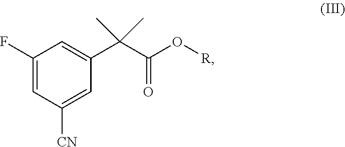

wherein R is C₁₋₄-alkyl, and
b) hydrolyzing the ester of formula III to afford 2-(3-cyano-5-fluorophenyl)-2-methylpropanoic acid (formula (I)).
2. The process of embodiment 1, wherein the palladium cross coupling catalyst is a Pd-trialkylphosphine complex catalyst.
3. The process of embodiment 2, wherein the palladium cross coupling catalyst is generated in situ from a Pd precursor and a trialkylphosphine.
4. The process of any one of embodiments 1 to 3, wherein the isobutyrate source is a C₁₋₄-alkyl isobutyrate or a silyl ketene acetal.
5. The process of any one of embodiments 1 to 4, wherein the isobutyrate source is methyl isobutyrate or methyl trimethylsilyl dimethylketene acetal.
6. The process of any one of embodiments 1 to 5, wherein the reaction of the palladium cross coupling catalyst with methyl isobutyrate takes place in the presence of a strong base.
7. The process of embodiment 6, wherein the strong base is a lithium dialkylamide.
8. The process of embodiment 6 or 7, wherein the reaction takes place in a non-polar organic solvent at a reaction temperature between −10° C. and 30° C.
9. The process of embodiment 4 or 5, wherein the reaction of the palladium cross coupling catalyst with the silyl ketene acetal takes place in the presence of a Zn compound as co-activator.
10. The process of embodiment 9, wherein the silyl ketene acetal is an alkyl trialkylsilyl dialkylketene acetal.
11. The process of embodiment 9 or 10, wherein the reaction takes place in a polar organic solvent at a reaction temperature between 40° C. and 100° C.
12. The process of any one of embodiments 1 to 11, wherein step b) is carried out with an alkali hydroxide.
13. The process of embodiment 12, wherein the alkali hydroxide is lithium hydroxide.
14. The process of embodiment 12 or 13, wherein the reaction takes place in the presence of a non-polar organic solvent at a reaction temperature between 0° C. and 100° C.
15. The process of any one of embodiments 1 to 11, wherein step b) is carried out with lithium iodide.
16. 2-(3-cyano-5-fluorophenyl)-2-methylpropanoic acid (formula (I)) produced according to any one of embodiments 1 to 15 for use in a method of preparing a compound of formula (VII), or a pharmaceutically acceptable salt thereof:

(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys20(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

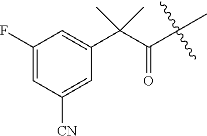 [SMILES: *C(=O)C(C)(C)c1cc(F)cc(C#N)c1]

and
AEEAc is 2-(2-(2-aminoethoxy) ethoxy) acetic acid.
17. A process for preparing a compound of formula (VII), or a pharmaceutically acceptable salt thereof:

(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-Trp²⁵-
Leu²⁶-Ile²⁷-Ala²⁸-Gly²⁹-Gly³⁰-Pro³¹-Ser³²-Ser³³-
Gly³⁴-Ala³⁵-Pro³⁶-Pro³⁷-Pro³⁸-Ser³⁹-NH₂,

wherein X is

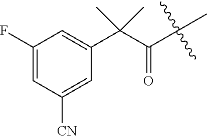 [SMILES: *C(=O)C(C)(C)c1cc(F)cc(C#N)c1]

and
AEEAc is 2-(2-(2-aminoethoxy) ethoxy) acetic acid,
the process comprising the use of 2-(3-cyano-5-fluorophenyl)-2-methylpropanoic acid (formula (I)).
18. A process for preparing a compound of formula (VII), or a pharmaceutically acceptable salt thereof:

(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-
Gln¹⁹-Lys²⁰(AEEAc-AEEAc-γ-Glu-19-
carboxynonadecanoyl)-Ala²¹-Phe²²-Val²³-Gln²⁴-
Trp²⁵-Leu²⁶-Ile²⁷-Ala²⁸-Gly²⁹-Gly³⁰-Pro³¹-Ser³²-
Ser³³-Gly³⁴-Ala³⁵-Pro³⁶-Pro³⁷-Pro³⁸-Ser³⁹-NH₂,

wherein X is

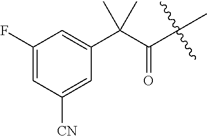 [SMILES: *C(=O)C(C)(C)c1cc(F)cc(C#N)c1]

and
AEEAc is 2-(2-(2-Aminoethoxy) ethoxy) acetic acid,
wherein the method comprises the steps of
a) synthesizing the compound of formula (VII) on a resin using solid phase synthesis methods, wherein the last step of the solid phase synthesis comprises adding the fragment X by coupling the resin-bound peptide with 2-(3-cyano-5-fluorophenyl)-2-methylpropanoic acid,
b) globally deprotecting and cleaving the compound of formula (VII) from the resin using an acid,
c) precipitating the compound of formula (VII) or salt thereof,
d) purifying the compound of formula (VII) or salt thereof, and
e) isolating the compound of formula VII or salt thereof.
19. The process of embodiment 17 or 18, wherein the solid phase synthesis of the compound of formula (VII) is performed on an amide resin selected from the group consisting of Rink amide resin, Sieber amide resin, and Ramage amide resin.
20. The process of embodiment 17 or 18, wherein the amide resin is a Rink amide resin or Sieber amide resin.
21. The process of any one of embodiments 17 to 19, wherein the solid phase synthesis comprises Fmoc deprotection.
22. The process of any one of embodiments 17 to 20, wherein the solid phase synthesis comprises sequential coupling of the following:
   (1) Fmoc-L-Ser(O′Bu)-OH,
   (2) Fmoc-L-Pro-OH,
   (3) Fmoc-L-Pro-OH,
   (4) Fmoc-L-Pro-OH,
   (5) Fmoc-L-Ala-OH,
   (6) Fmoc-Gly-OH,
   (7) Fmoc-L-Ser(O′Bu)-OH,
   (8) Fmoc-L-Ser(O′Bu)-OH,
   (9) Fmoc-L-Pro-OH,
   (10) Fmoc-Gly-Gly-OH,
   (11) Fmoc-L-Ala-OH,
   (12) Fmoc-L-Ile-OH,
   (13) Fmoc-L-Leu-OH,
   (14) Fmoc-L-Trp (Boc)-OH,
   (15) Fmoc-L-Gln(Trt)-OH,
   (16) Fmoc-L-Val-OH,
   (17) Fmoc-L-Phe-OH,
   (18) Fmoc-L-Ala-OH,
   (19) ivDde-L-Lys(Fmoc)-OH,
   (20) Fmoc-AEEA-OH,
   (21) Fmoc-AEEA-OH,
   (22) Fmoc-L-Glu-O′Bu,
   (23) 20-(tert-butoxy)-20-oxoicosanoic acid, followed by deprotection of ivDde with 3% v/v hydrazine monohydrate/DMF,
   (24) Fmoc-L-Gln(Trt)-OH,
   (25) Fmoc-L-Ala-OH,
   (26) Fmoc-L-Ile-OH,
   (27) Fmoc-L-Lys(Boc)-OH,
   (28) Fmoc-L-Asp(O′Bu)-OH,
   (29) Fmoc-L-Leu-OH,
   (30) Fmoc-Aib-OH,
   (31) Fmoc-L-Ile-OH,
   (32) Fmoc-L-Ser(O′Bu)-OH,
   (33) Fmoc-L-Tyr(O′Bu)-OH,
   (34) Fmoc-L-Asp(O′Bu)-OH,
   (35) Fmoc-L-Ser(O′Bu)-OH,
   (36) Fmoc-L-Thr(O′Bu)-OH,
   (37) Fmoc-L-Phe-OH,
   (38) Fmoc-L-Thr(O′Bu)-OH,
   (39) Fmoc-L-Gly-OH,
   (40) Fmoc-L-Glu (O′Bu)-OH, and
   (41) Fmoc-β-Ala-OH, and
   (42) 2-(3-cyano-5-fluorophenyl)-2-methylpropanoic acid.
23. The process of any one of embodiments 17 to 20, wherein the solid phase synthesis comprises pseudoproline building blocks.
24. The process of any one of embodiments 17 to 20 or 22, wherein the solid phase synthesis comprises sequential coupling of the following:
   (1) Fmoc-L-Ser(O′Bu)-OH,
   (2) Fmoc-L-Pro-OH,
   (3) Fmoc-L-Pro-L-Pro-OH,
   (4) Fmoc-L-Ala-OH·H₂O,
   (5) Fmoc-Gly-OH,
   (6) Fmoc-L-Ser(O′Bu)-OH,
   (7) Fmoc-L-Ser(O′Bu)-OH,
   (8) Fmoc-L-Pro-OH·H₂O,
   (9) Fmoc-Gly-Gly-OH,
   (10) Fmoc-L-Ala-OH,
   (11) Fmoc-L-Ile-OH,
   (12) Fmoc-L-Leu-OH,
   (13) Fmoc-L-Trp (Boc)-OH,
   (14) Fmoc-L-Gln(Trt)-OH,
   (15) Fmoc-L-Val-OH,
   (16) Fmoc-L-Phe-OH,
   (17) Fmoc-L-Ala-OH,
   (18) ivDde-L-Lys(Fmoc)-OH,
   (19) Fmoc-AEEA-OH,
   (20) Fmoc-AEEA-OH,
   (21) Fmoc-L-Glu-O′Bu,
   (22) 20-(tert-butoxy)-20-oxoicosanoic acid, followed by deprotection of ivDde with 3% v/v hydrazine monohydrate/DMF,
   (23) Fmoc-L-Gln(Trt)-OH,
   (24) Fmoc-L-Ala-OH,
   (25) Fmoc-L-Ile-OH,
   (26) Fmoc-L-Lys(Boc)-OH,
   (27) Fmoc-L-Asp(O′Bu)-OH,
   (28) Fmoc-L-Leu-OH,
   (29) Fmoc-Aib-OH,
   (30) Fmoc-L-Ile-OH,
   (31) Fmoc-L-Tyr(′Bu)-L-Ser(Ψ(Me,Me)pro)-OH,
   (32) Fmoc-L-Asp(O′Bu)-OH,
   (33) Fmoc-L-Ser(O′Bu)-OH,
   (34) Fmoc-L-Thr(O′Bu)-OH,
   (35) Fmoc-L-Phe-OH,
   (36) Fmoc-Gly-L-Thr(Ψ(Me,Me)pro)-OH,
   (37) Fmoc-L-Glu (O′Bu)-OH,
   (38) Fmoc-β-Ala-OH, and
   (39) 2-(3-cyano-5-fluorophenyl)-2-methylpropanoic acid.
25. The process of any one of embodiments 18 to 23, wherein the acid used in the global deprotection and cleavage step is trifluoroacetic acid (TFA).
26. The process of any one of embodiments 18 to 24, wherein the global deprotection and cleavage step is carried out with a cleavage mixture comprising trifluoroacetic acid (TFA) in combination with a scavenger.
27. The process of embodiment 25 or 26, wherein the global deprotection and cleavage step is carried out with a cleavage mixture comprising TFA, tri-isopropyl-silane (TIS), and water.
28. The process of any one of embodiments 18 to 27, wherein the precipitating step comprises use of an anti-solvent, optionally wherein the anti-solvent is methyl t-butyl ether (TBME).
29. The process of any one of embodiments 18 to 27, wherein the purifying step comprises use of a preparative reversed phase HPLC purification.
30. The process of any one of embodiments 18 to 29, wherein the purifying step further comprises a desalting step.
31. The process of any one of embodiments 18 to 30, wherein the isolating step comprises
   (i) a solvent/anti-solvent precipitation process,
   (ii) a spray drying process, or
   (iii) freeze-drying.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Any compound disclosed herein can be used in any of the treatment method here, wherein the individual to be treated is as defined anywhere herein. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

The following Examples may use the abbreviations set out in Table 1.

TABLE 1
Abbreviations

| | |
|---|---|
| AcOH | acetic acid |
| DIC | N,N′-diisopropylcarbodiimide |
| DMF | N,N-dimethylformamide |
| DVB | divinylbenzene |
| DODT | 2,2′-(ethylenedioxy)diethanethiol |
| DTE | 1,4-dithioerythritol |
| DTT | 1,4-dithiothreitol |
| EDT | 1,2-dithioethane |
| EtOAc | ethylacetate |
| FMOC | fluorenylmethyloxycarbonyl |
| HNCy₂ | dicyclohexylamine |
| HOBT | 1-hydroxybenzotriazole |
| ivDde | 1-(4,4-dimethyl-2,6-dioxocyclohexylidene)-3- |
| | methylbutyl |
| i-PrOAc | isopropyl acetate |
| MBHA Resin | Rink Amide MBHA (4-methylbenzhydrylamine) Resin |
| MeCN | acetonitrile |
| MeOH | methanol |
| MTBE | methyl-t-butylether |
| NMP | N-methyl-2-pyrrolidone |
| n-BuLi | n-butyl lithium |
| OxymaPure | ethyl cyano(hydroxyimino)acetate |
| PAd₃ | tris(1-adamantyl)phosphine |
| [Pd(allyl)Cl]₂ | allylpalladium(II)-chloride dimer |
| Pd(dppf)Cl₂ | [1,1′-Bis(diphenylphosphino)ferrocen]dichlor- |
| | palladium(II) |
| Pd₂dba₃ | tris(dibenzylidenacetone)dipalladium(0) |
| PE | petroleum ether |
| [Pt-Bu₃)PdBr]₂ | Bis (tri-t-butylphosphine) palladium bromide (I), dimer |
| 2-PrOH | 2-propanol |
| RT | room temperature |
| Sieber Resin | 9-Fmoc-aminoxanthen-3-yloxy resin |
| Silica-SH | thiol (SH)-functionalized silica gel (e.g. Sorbtech Catalog |
| | No. 40930M85) |
| t-Bu₃P•HBF₄ | tri-t-butylphosphoniumtetrafluoroborate |
| TBME | methyl t-butyl ether |
| TFA | trifluoroacetic acid |
| THF | tetrahydrofuran |
| TIS | tri-isopropyl-silane |

Examples 2 and 3 follow the general synthetic scheme set forth below.

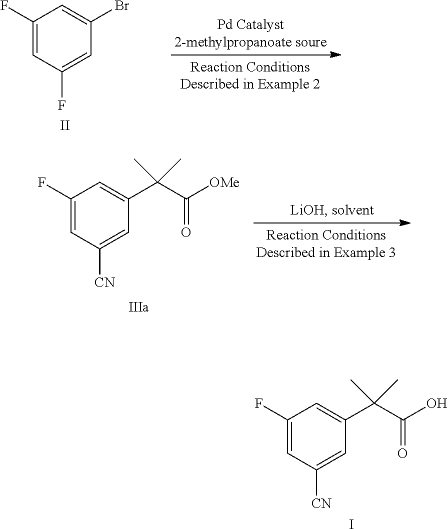

### Example 1: Preparation of 2-(3-cyano-5-fluorophenyl)-2-methylpropanoic Acid

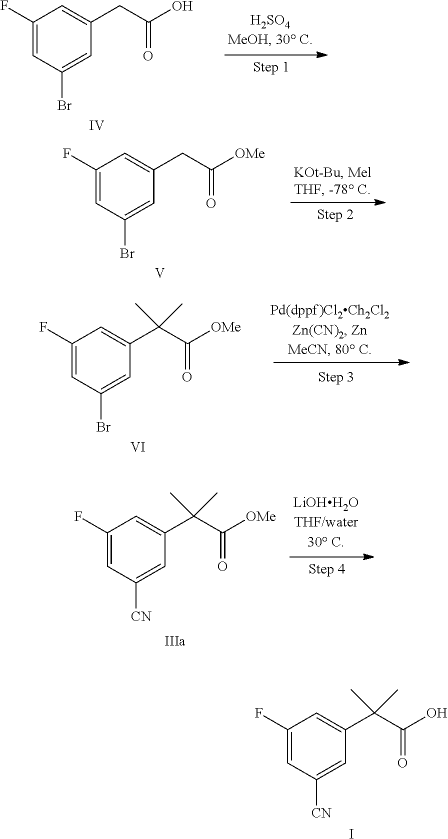

Step 1: To a solution of 2-(3-bromo-5-fluorophenyl) acetic acid (100.0 g, 429.1 mmol, 1.00 equiv.) in MeOH (1000 mL) was added sulfuric acid (8.42 g, 85.8 mmol, 0.20 equiv.) at 30° C. and the resulting reaction mixture was stirred. After complete conversion, the reaction mixture was concentrated under reduced pressure to approx. 3-4 volumes and quenched by addition of water (200 mL). 5% aqueous NaHCO₃ solution (1000 mL) was then added and the reaction mixture was extracted with EtOAc (1200 mL). After phase separation, the aqueous layer was re-extracted with EtOAc (1000 mL). The combined organic extracts were sequentially washed with water (1000 mL) and 25% aqueous NaCl solution (1000 mL), dried with Na₂SO₄, filtered, and concentrated under reduced pressure to obtain crude methyl 2-(3-bromo-5-fluorophenyl)acetate (V) as a colorless oil (103 g, 97.2% yield, HPLC purity: 98.8% area). 1H NMR (300 MHz, CDCl₃): δ 7.25 (d, 1H), 7.17 (d, 1H), 6.97 (d, 1H), 3.72 (s, 3H), 3.59 (s, 2H).

Step 2: A solution of methyl 2-(3-bromo-5-fluorophenyl)acetate (V) (50.00 g, 202.4 mmol, 1.00 equiv.) in THF (600 mL) was cooled to −78° C. A solution of KOt-Bu (1 M in THF, 401.0 g, 444.4 mmol, 2.20 equiv.) was added at this temperature followed by methyl iodide (115.0 g, 810.2 mmol, 4.00 equiv.). The reaction mixture was stirred at −78° C. for 2 hours then allowed to warm slowly to ambient temperature. After complete conversion, the reaction mixture was quenched with water (1000 mL), followed by addition of EtOAc (1000 mL). After phase separation, the aqueous layer was extracted with EtOAc (2×500 mL). The combined organic extracts were washed with 25% aqueous NaCl solution (2×500 mL).

After phase separation, the organic layer was dried (Na₂SO₄), filtered and concentrated under reduced pressure to obtain crude methyl 2-(3-bromo-5-fluorophenyl)-2-methylpropanoate (VI) as colorless oil (50.0 g, 89.8% yield, HPLC purity: 95.3% area). 1H NMR (300 MHz, CDCl₃): δ 7.26 (s, 1H), 7.15 (d, 1H), 6.97 (d, 1H), 3.68 (s, 3H), 1.56 (s, 6H).

Step 3: A solution of methyl 2-(3-bromo-5-fluorophenyl)-2-methylpropanoate (VI) (300.0 g, 1.09 mol, 1.00 equiv.) in MeCN (6000 mL) was prepared. Zn powder (71.4 g, 1.09 mol, 1.00 equiv.), Pd (dppf)Cl₂·CH₂Cl₂ (178.2 g, 218.2 mmol, 0.20 equiv.) and Zn(CN)₂ (90.00 g, 766.3 mmol, 0.70 equiv.) were added to the solution, then the reaction mixture was heated to 80-85° C. and stirred at this temperature. After complete conversion, the reaction mixture was cooled to ambient temperature and filtered through a bed of diatomaceous earth. The filtrate was concentrated under reduced pressure to approximately 2-3 volumes and diluted with MTBE (4500 mL). The resulting mixture was filtered through a bed of diatomaceous earth. The filtrate was washed with 5% aqueous sodium thiosulfate solution (3000 mL). After phase separation, the aqueous layer was re-extracted with MTBE (3000 mL). The combined organic extracts were washed with 25% aqueous NaCl solution (3000 mL).

After phase separation, the organic layer was treated with activated carbon and filtered through a bed of diatomaceous earth. The filtrate was dried (Na₂SO₄), filtered and concentrated under reduced pressure to obtain crude methyl 2-(3-cyano-5-fluorophenyl)-2-methylpropanoate (IIIa) as colorless oil (230 g, 95.2% yield, HPLC purity: 84.0% area). 1H NMR (300 MHz, CDCl₃): δ 7.45 (s 1H), 7.33-7.24 (m, 2H), 3.69 (s, 3H), 1.60 (s, 6H).

Step 4: A solution of methyl 2-(3-cyano-5-fluorophenyl)-2-methylpropanoate (IIIa) (200.0 g, 904.0 mmol, 1.00 eq) in THF (4000 mL) was prepared and cooled to 0° C. A solution of LiOH. H₂O (49.40 g, 1.18 mol, 1.30 equiv.) in water (2000 mL) was added at. The reaction mixture was then warmed to 30° C. and stirred at this temperature. After complete conversion, EtOAc (2000 mL) was added. The phases were separated and the aqueous layer was washed with EtOAc (2000 mL). The combined organic layers were extracted with water (2×1000 mL). The aqueous layers were combined, and the pH was adjusted to 3 by addition of 10% acetic acid solution (3000 mL). The product was extracted to the organic layer with EtOAc (2×2000 mL). The combined organic extracts were sequentially washed with water (2000 mL) and 25% aqueous NaCl solution (2000 mL).

The organic layer was treated with activated carbon and filtered over a bed of diatomaceous earth. The filtrate was dried (using Na₂SO₄), filtered and concentrated under reduced pressured to 1-2 volumes. The residue was stripped with n-heptane (2×400 mL) before additional n-heptane (600 mL) was added at 30° C. The resulting suspension was maintained at 30° C. for one hour. The solids were filtered off, washed with n-heptane (400 mL) and dried at 30° C. The solids were dissolved in DMF (400 mL) at 30° C. and water (1000 mL) was added slowly to precipitate the compound. The solids were filtered off and washed with water (1000 mL). The wet material was slurried in water (2000 mL), filtered, sequentially washed with water (1000 mL) and n-heptane (600 mL), and dried at 50° C. 2-(3-Cyano-5-fluorophenyl)-2-methylpropanoic acid (I) was obtained as a white powder (140 g, 74.7% yield, HPLC purity: 99.2% area). 1H-NMR (300 MHz, DMSO-d6): δ 12.69 (s, 1H), 7.77-7.54 (m, 3H), 1.51 (s, 6H).

### Example 2: Preparation of Methyl 2-(3-cyano-5-fluorophenyl)-2-methylpropanoate)

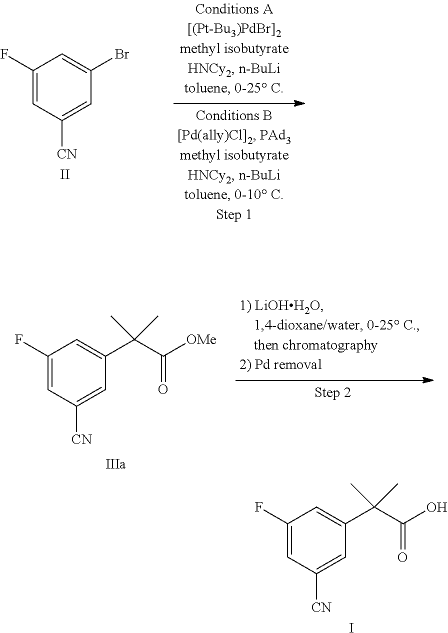

### [(Pt-Bu3)PdBr]₂ Route; Step 1—Conditions A

Dicyclohexylamine (198.3 g, 217.9 mL, 1.09 mol, 1.75 equiv.) was dissolved in toluene (2500 mL) and the resulting solution was cooled to 0° C. n-BuLi (2.5 M in hexane, 389.9 mL, 974.8 mmol, 1.56 equiv.) was added and the resulting reaction mixture was stirred at 0° C. Methyl 2-methylpropanoate (95.7 g, 107.5 mL, 937.5 mmol, 1.50 equiv.) was then added at 0° C. and the reaction mixture was stirred at this temperature to give a clear solution. Then, 3-bromo-5-fluorobenzonitrile (125.0 g, 625.0 mmol, 1.00 equiv.) and [(Pt-Bu₃)PdBr]₂ (4.86 g, 6.25 mmol, 0.01 eq) were added. The reaction mixture was heated to 25° C. and stirred at this temperature. After completed conversion, the reaction mixture was diluted with MTBE (2100 mL), then cooled to 5° C. and quenched with 1 M HCl (1800 mL) to adjust the pH to 4. The resulting mixture was then stirred at 25° C. The resulting precipitate was removed by filtration and the filter cake was washed with MTBE (4×300 mL). The phases of the filtrate were separated. The organic phase was sequentially washed with sat. aq. NaHCO₃ solution (500 mL) and saturated aqueous NaCl solution (500 mL), dried (Na₂SO₄), filtered and concentrated under reduced pressure.

The crude material was purified by column chromatography (SiO₂, EtOAc in PE from 0% to 3%) to give crude methyl 2-(3-cyano-5-fluorophenyl)-2-methylpropanoate (IIIa) (59.4 g, 43.0% yield) as a yellow oil. 1H NMR (500 MHz, CDCl₃): δ 7.44 (t, J=1.6 Hz, 1H), 7.31 (dt, J=10.0, 2.1 Hz, 1H), 7.26-7.23 (m, 1H), 3.69 (s, 3H), 1.59 (s, 6H). ¹⁹F NMR (471 MHz, CDCl₃): δ −109.38. GC-MS (EI+): mass calculated for C₁₂H₁₂FNO₂ [M]⁺ 221.1, found 221.1.

### [Pd(allyl)Cl]₂ Route; Step 1—Conditions B

A mixture of [Pd(allyl)Cl]₂ (366.0 mg, 1.00 mmol, 1.00 mol %) and PAd₃ (960.0 mg, 2.20 mmol, 2.20 mol %) in toluene (20 mL) was stirred at ambient temperature overnight. In a separate vessel, n-BuLi (2.5 M in hexane, 60.0 mL, 150.0 mmol, 1.50 equiv.) was added slowly to a solution of HNCy₂ (31.9 mL, 160.0 mmol, 1.60 equiv.) in toluene (180 mL) at 0° C. and the resulting mixture was stirred at 0° C. for 30 min. Methyl isobutyrate (20.6 mL, 180.0 mmol, 1.80 equiv.) was added at 0° C. and stirring continued at 0° C. for 30 minutes. Then, 3-bromo-5-fluorobenzonitrile (20.00 g, 100.0 mmol, 1.00 equiv.) and the above catalyst suspension were added at 0° C. and the resulting reaction mixture was stirred at 10° C. After complete conversion, the reaction mixture was diluted with i-PrOAc (336 mL) and 1 M aqueous HCl solution (288 mL) was added to adjust the pH to 4. The resulting suspension was filtered, the filter cake was washed with i-PrOAc (4×48 mL), and the phases of the filtrate were separated. The organic layer was sequentially washed with saturated aqueous NaHCO₃ solution (80 mL) and saturated aqueous NaCl solution (20 mL). The organic layer was filtered through a plug of activated carbon. The filtrate was concentrated under reduced pressure and stripped with THF (2×1−2 vol.). The residue was taken up in THF (13-15 volumes). Methyl 2-(3-cyano-5-fluorophenyl)-2-methylpropanoate (IIIa) was obtained as THF solution (17.7 g net amount of the target compound, 80% yield) which was telescoped into the next step (Example 2.3).

Pd₂(dba₃) Route

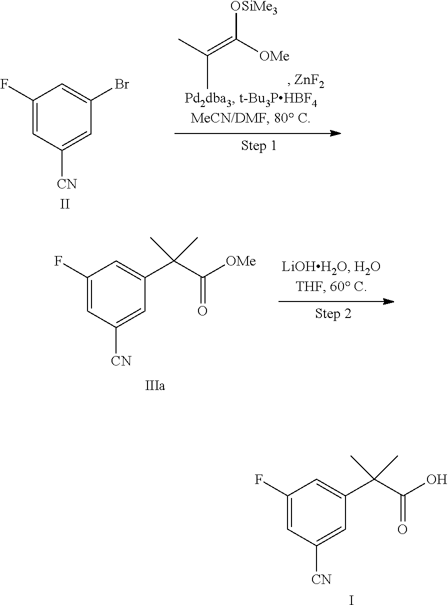

To a solution of 3-bromo-5-fluorobenzonitrile (100.0 g, 500.0 mmol, 1.00 equiv.) and methyl trimethylsilyl dimethylketene acetal (174.3 g, 1.00 mol, 2.00 equiv.) in DMF (500 mL) and MeCN (1500 mL) was added ZnF₂ (52.2 g, 504.8 mmol, 1.01 equiv.), Pd₂dba₃ (4.60 g, 5.02 mmol, 1.00 mol %) and 1-Bu₃P·HBF₄ (5.82 g, 20.6 mmol, 4.00 mol %). The mixture was heated to 80° C. and stirred at this temperature. After complete conversion, the reaction mixture was filtered over a plug of diatomaceous earth and the filter cake was washed with DMF/MeCN (1:3 v/v, 200 mL). The filtrate was concentrated under reduced pressure and the residue was taken up in i-PrOAc (1000 mL). The organic layer was washed with water (500 mL; 10% citric acid was added until complete dissolution of all solids). The phases were separated and the organic layer was washed with 5% aqueous LiCl solution (2×500 mL) before it was filtered through a plug of activated carbon. The filtrate was concentrated under reduced pressure and stripped with THF (2×1−2 vol.). The residue was taken up in THF (13-15 volumes). Methyl 2-(3-cyano-5-fluorophenyl)-2-methylpropanoate (IIIa) was obtained as THF solution (99.5 g net amount of the target compound, 90% yield) which was telescoped into the next step.

### Optimization of Reaction Conditions

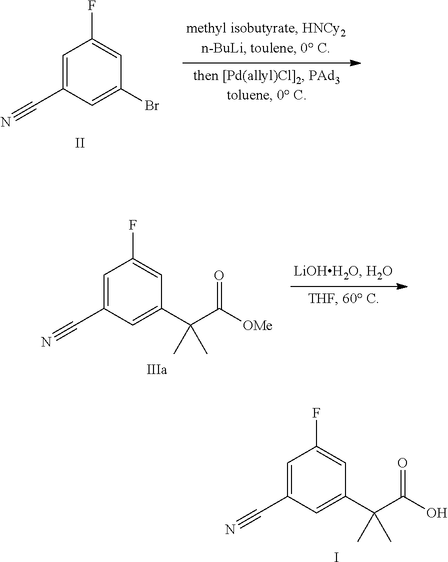

A mixture of HNCy₂ (118 g, 0.65 mol, 1.3 equiv.) and toluene (900 mL, 9 vol.) was sparged with nitrogen for 30 min at ambient temperature. The mixture was then cooled to 0° C. and n-BuLi (2.5 M in n-hexane, 240 mL, 0.60 mol, 1.2 equiv.) was added dropwise over 5 minutes. The resulting mixture was stirred at 0° C. for 30 minutes before methyl isobutyrate (61 g, 0.60 mol, 1.2 equiv) was added and the reaction stirred for a further 30 min at 0° C. Then, 3-bromo-5-fluorobenzonitrile (100 g, 0.50 mol, 1.0 equiv.) and PAd₃ (4.8 g, 11 mmol, 2.2 mol %) were added and the mixture was sparged with nitrogen for 10 min at 0° C. A solution of [Pd(allyl)Cl]₂ (1.8 g, 5.0 mmol, 1.0 mol %) in toluene (300 mL, 3 vol.) was added at 0° C. and the resulting reaction mixture was stirred. Upon completion of the reaction, water (500 mL, 5 vol.) was added, and the mixture was filtered through diatomaceous earth at 0° C. The phases were separated, and the organic phase was sequentially washed with 15% aqueous citric acid solution (600 mL, 6 volumes) and water (2×500 mL, 2×5 volumes). The organic phase was then filtered through activated carbon and the filter cake was washed with toluene (300 mL, 3 volumes). The filtrate was concentrated under reduced pressure to 1-2 volumes. Then, THF (300 mL, 3 vol.) was added and methyl 2-(3-cyano-5-fluorophenyl)-2-methylpropanoate (IIIa) was obtained as THF solution (94.9 g net amount of the target compound, 85.8% yield), which was used directly in the next step (assuming 100% yield).

THF (1.0 L, 9 vol.) was added to the THF solution containing methyl 2-(3-cyano-5-fluorophenyl)-2-methylpropanoate (IIIa). LiOH·H₂O (29.4 g, 0.70 mol, 1.4 equiv.) and water (18 g, 1.0 mol, 2.0 equiv.) were then added at ambient temperature before the reaction mixture was heated to 60° C. and stirred. Upon completion of the reaction, the mixture was concentrated under reduced pressure to six volumes. Toluene (550 mL, 5 vol.) was added, and the mixture was again concentrated under reduced pressure to six volumes before it was cooled to ambient temperature. 10% aqueous citric acid solution (110 g, 100 weight %) and water (500 mL, 4.5 vol.) were added to adjust the pH to 9.5-10.5 and the resulting mixture was stirred at ambient temperature for one hour. The phases were separated and the organic phase was discarded. Toluene/THF (4.5:1 v/v, 1.1 L, 10 vol.) was added to the aqueous phase and the pH was adjusted to 5.3-6.0 by the addition of 10% aqueous citric acid solution (330 mL, 3 vol.). The resulting mixture was stirred for one hour before the phases were separated. The aqueous phase was again extracted with toluene/THF (4.5:1 v/v, 2×550 mL, 2×5 vol.). The combined organic phases were washed with water (550 mL, 5 vol.) and filtered through activated carbon. The filter cake was washed with toluene/THF (4.5:1 v/v, 220 mL, 2 vol.) and the filtrate was concentrated under reduced pressure. Toluene (330 mL, 3 vol.) was added, and the mixture was again concentrated under reduced pressure. Toluene (330 mL, 3 vol.) was added, and the mixture was concentrated under reduced pressure to 1.0-2.0 volumes before another portion of toluene (170 mL, 1.5 vol.) was added. The resulting mixture was heated to 65° C. and stirred at this temperature for one hour to obtain a clear solution. The solution was cooled to 55-60° C. and seed crystals were added. Stirring continued at this temperature for one hour, followed by cooling the mixture to 0° C. The resulting solid was isolated by filtration. The filter cake was washed with toluene (110 mL, 1 volumes) and dried under reduced pressure to afford 2-(3-cyano-5-fluorophenyl)-2-methylpropanoic acid (I) (71.2 g, 68.8% yield over two steps) as a white solid.

### Example 3: Preparation of 2-(3-cyano-5-fluorophenyl)-2-methylpropanoic Acid

### LiOH·H₂O/Dioxane/Aqueous Solution

To a solution of methyl 2-(3-cyano-5-fluorophenyl)-2-methylpropanoate (IIIa) (130.0 g, 587.6 mmol, 1.00 equiv.) in 1,4-dioxane (1820 mL), a solution of LiOH·H₂O (25.90 g, 617.0 mmol, 1.05 equiv.) in water (910 mL) was added at 0° C. The reaction mixture was slowly heated to 25° C. and stirred at this temperature. After complete conversion, the reaction mixture was quenched with 1 M aqueous HCl solution (900 mL) at 0° C. and extracted with EtOAc (3×1000 mL). The combined organic layers were dried (Na₂SO₄), filtered and concentrated under reduced pressure. The crude material was purified by column chromatography (SiO₂, EtOAc in (PE:AcOH=1000:1) from 0% to 18%) to give 2-(3-cyano-5-fluorophenyl)-2-methylpropanoic acid (I) (99.0 g, 81.4% yield) as a white solid.

LiOH·H₂O/1.5 equiv. Water/THF

LiOH·H₂O (32.26 g, 768.7 mmol, 1.70 equiv.) and water (12.22 g, 678.4 mmol, 1.50 equiv.) were added to the reaction solution obtained from Example 2.3. The reaction mixture was warmed to 60° C. and stirred. Upon complete conversion, the reaction mixture was cooled to ambient temperature and the pH was adjusted to 4 by addition of 10% aqueous citric acid solution (8-10 vol.). The resulting mixture was extracted with i-PrOAc (700 mL). The phases were separated and the aqueous layer was re-extracted with i-PrOAc (2×500 mL). The combined organic layers were washed with 10% aqueous NaCl solution (500 mL) and then concentrated under reduced pressure to 10 volumes. To the residue was added KH₂PO₄/K₂HPO₄ buffer solution (500 mL) and the pH was adjusted to 7 by addition of 10% aqueous KOH solution and 10% aqueous citric acid solution. The phases were separated and the aqueous layer was extracted with i-PrOAc (4×500 mL) at pH 7. The combined organic layers were washed with 10% aqueous NaCl solution (500 mL) and treated with activated carbon. After filtration, the solvent was exchanged to toluene by distillation and the product was crystallized from toluene. After filtration, washing and drying, 2-(3-cyano-5-fluorophenyl)-2-methylpropanoic acid (I) was isolated as a white solid (70.5 g, 75.3% yield).

### Example 4: Removal of Pd from Crude 2-(3-cyano-5-fluorophenyl)-2-methylpropanoic Acid

A mixture of 2-(3-cyano-5-fluorophenyl)-2-methylpropanoic acid (325.0 g, 1.57 mol, 1.00 eq) in PE (3000 mL) was stirred at 25° C. for 16 hours. The reaction mixture was filtered and washed with PE (1500 mL).

The wet cake was taken up in PE (1560 mL) and heated to 60° C. Silica-SH (62.40 g, 20% weight) was added. The resulting mixture was stirred at 60° C. for 16 hours before being filtered at 60° C. The filter cake was washed with EtOAc/PE (3:5 v/v, 3×600 mL). The filtrate was concentrated under reduced pressure to give 2-(3-cyano-5-fluorophenyl)-2-methylpropanoic acid (I) as a white solid.

The above solid was taken up in n-heptane (2850 mL). The resulting suspension was stirred at 25° C. for 16 hours. The solids were filtered, washed with n-heptane (1500 mL) and dried at 40-50° C. to yield 2-(3-cyano-5-fluorophenyl)-2-methylpropanoic acid (I) (318.5 g, 97.8% yield) as a white solid. 1H NMR (500 MHZ, CDCl₃) δ 7.52 (t, J=1.5 Hz, 1H), 7.40 (dt, J=10.0, 2.1 Hz, 1H), 7.24-7.33 (m, 1H), 1.65 (s, 6H). ¹⁹F NMR (471 MHz, DMSO-d6) δ−109.10. GC-MS (EI+): mass calculated for formula C₁₁H₁₀FNO₂ [M]⁺ 207.1, found 207.0.

### Example 5: Solid-Phase Synthesis of the Compound of Formula (VII) Using Pseudoprolines

Preparation of the Resin: Fmoc Sieber Resin (648 g, loading 0.71 mmol/g, 460 mmol) was charged into a 20 L SPPS reactor. The resin was swelled with DMF (12.96 L, 20 mL/g resin) and stirred for two hours at ambient temperature. The reactor was then drained and the resin was washed twice with DMF (2×6.48 L, 10 mL/g resin).

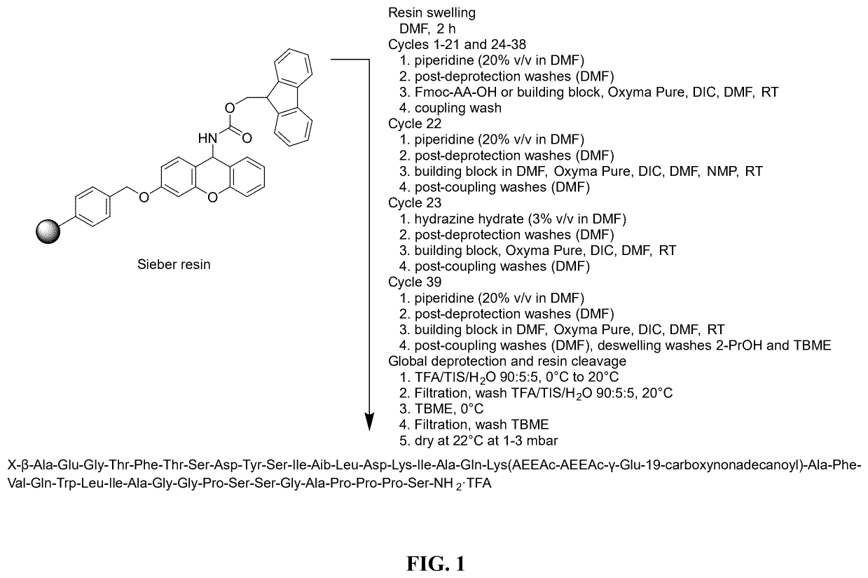

General Synthetic Procedure (Table 2, FIG. 1): Washing steps were designed to remove remaining deprotection solution, coupling reagents, or additives between process steps. Before each wash, the reactor was completely drained (i.e., until the flow into the waste accumulation vessel has stopped). The wash solvent was added to the reactor and the mixture was stirred for 5 minutes (starting after solvent addition is complete and stirring is started). The stirrer was then stopped and the reactor was drained completely.

Fmoc deprotection was performed by stirring the resin in Fmoc deprotection solution. For all deprotections except for the deprotections in cycles 19 and 23, deprotection treatments were performed 3 times with a stirring time of 10 minutes for each treatment. Time starts after solvent addition is complete and stirring is started. The Fmoc deprotection solution consists of a 20% v/v piperidine solution in DMF. Fmoc deprotection in cycle 19 was performed 5 times.

In cycle 23, ivDde deprotection was performed by treatment with a 3% v/v solution of hydrazine monohydrate in DMF for 16 hours.

TABLE 2
Building Blocks Coupled in Sold-Phase Peptide Synthesis
Amino acid / building block coupled (SEQ ID NO: 2)

| 1 | 2 | 3 | 4 | 5 | 6 |
|---|---|---|---|---|---|
| Ser³⁹ | Pro³⁸ | Pro³⁷-Pro³⁶ | Ala³⁵ | Gly³⁴ | Ser³³ |
| 7 | 8 | 9 | 10 | 11 | 12 |
| Ser³² | Pro³¹ | Gly³⁰-Gly²⁹ | Ala²⁸ | Ile²⁷ | Leu²⁶ |
| 13 | 14 | 15 | 16 | 17 | 18 |
| Trp²⁵ | Gln²⁴ | Val²³ | Phe²² | Ala²¹ | Lys²⁰ |
| 19 | 20 | 21 | 22 | 23 | 24 |
| AEEAc^{20.1} | AEEAc^{20.2} | gGlu²⁰ | C₂₀ acid^{20.4} | Gln¹⁹ | Ala¹⁸ |
| 25 | 26 | 27 | 28 | 29 | 30 |
| Ile¹⁷ | Lys¹⁶ | Asp¹⁵ | Leu¹⁴ | Aib¹³ | Ile¹² |
| 31 | 32 | 33 | 34 | 35 | 36 |
| Tyr¹⁰- | Asp⁹ | Ser⁸ | Thr⁷ | Phe⁶ | Gly⁴- |
| Ser(Ψ(Me,Me)pro)¹¹ | | | | | Thr(Ψ(Me,Me)pro)⁵ |

| 37 | 38 | 39 |
|---|---|---|
| Glu³ | β-Ala² | X¹ = 2-(3-cyano-5-fluorophenyl)-2-methylpropanoic acid |

For all couplings, a DIC/OxymaPure procedure was used. The protected amino acid or building block and OxymaPure were dissolved together in DMF (except in cycle 22, where the building block and OxymaPure were dissolved in NMP). As the resin volume increases with the progressing SPPS build, the later coupling steps were carried out under more diluted conditions. A solution of DIC in DMF was then added and the resulting mixture was stirred at ambient temperature for 2 minutes to pre-activate the amino acid.

Once pre-activation was complete, the amino acid solution was added to the reactor. The reaction mixture was then stirred. The reaction time for the coupling was 1.5-22 hours. Recoupling was performed for cycles 21, 30, 37 and 38 using the same procedure as for the coupling.

In-process monitoring was carried out following selected deprotections and couplings. Upon completion of the coupling sequence the fragment was cleaved from the resin and conversion was assessed by HPLC.

Synthetic Procedure: Starting with the Fmoc-deprotection of the protected resin, the peptide backbone and the side chain on Lys²⁰ were built using the general conditions for each amino acid coupling and deprotection outlined in Table 3.

TABLE 3
Conditions Used for the SPPS build

| Entry | Condition/Parameter | Build | |
|---|---|---|---|
| 1 | Initial Sieber resin loading | 0.71 | mmol/g |
| 2 | Amount DMF for each post-coupling wash | 10 | mL/g resin |
| 3 | Amount piperidine in DMF (20% v/v) for each | 10 | mL/g resin |
| | Fmoc deprotection treatment | | |
| 4 | Amount N₂H₄•H₂O in DMF (3% v/v) for each | 10 | mL/g resin |
| | ivDde deprotection treatment | | |
| 5 | Amount DMF for each post-deprotection wash | 10 | mL/g resin |
| 6 | Amount 2-PrOH for each de-swelling wash | 10 | mL/g resin |
| 7 | Amount TBME for each de-swelling wash | 10 | mL/g resin |
| 8 | Pre-activation time for all couplings | 2 | min |

| | | | |
|---|---|---|---|
| 9 | Reagents used for coupling | 1-21 | 2 equiv Fmoc-AA-OH or |
| | in cycles | and | building block |
| | | 23-38 | 2 equiv OxymaPure |
| | | | 3 equiv DIC |
| | | | DMF solvent |
| | | 22 | 2 equiv building block |
| | | | 2 equiv OxymaPure |
| | | | 3 equiv DIC |
| | | | DMF and NMP solvents |
| | | 9 | 1.87 equiv 2-(3-cyano-5- |
| | | | fluorophenyl)-2- |
| | | | methylpropanoic acid |
| | | | 2 equiv OxymaPure |
| | | | 3 equiv DIC |
| | | | DMF solvent |
| 10 | Volume of coupling | 1-21 | 5.68 mL/g of initial resin |
| | solution excluding | and | charge |
| | OxymaPure and building | 23-30 | (2677 mL DMF used for |
| | blocks used for coupling | | dissolving the protected amino |
| | in cycles | | acid or building block and |
| | | | OxymaPure, 399.3 mL DMF |
| | | | and 213.7 mL DIC used to |
| | | | make the DIC/DMF solution, |
| | | | 400 mL DMF directly charged |
| | | | into the reactor) |
| | | 22 | 5.68 mL/g of initial resin |
| | | | charge |
| | | | (2677 mL NMP used for |
| | | | dissolving the protected |
| | | | building block and |
| | | | OxymaPure, 399.3 mL DMF |
| | | | and 213.7 mL DIC used to |
| | | | make the DIC/DMF solution, |
| | | | 400 mL DMF directly charged |
| | | | into the reactor) |
| | | 31-35 | 6.06 mL/g of initial resin |
| | | | charge |
| | | | (2917 mL DMF used for |
| | | | dissolving the protected amino |
| | | | acid and OxymaPure, 399.3 |
| | | | mL DMF and 213.7 mL DIC |
| | | | used to make the DIC/DMF |
| | | | solution, 400 mL DMF directly |
| | | | charged into the reactor) |
| | | 36-38 | 6.58 mL/g of initial resin |
| | | | charge |
| | | | (3250 mL DMF used for |
| | | | dissolving the protected amino |
| | | | acid and OxymaPure, 399.3 |
| | | | mL DMF and 213.7 mL DIC |
| | | | used to make the DIC/DMF |
| | | | solution, 400 mL DMF directly |
| | | | charged into the reactor) |
| | | 39 | 8.0 mL/g of initial resin charge |
| | | | (4167 mL DMF used for |
| | | | dissolving the building block |
| | | | and OxymaPure, 399.3 mL |
| | | | DMF and 213.7 mL DIC used |
| | | | to make the DIC/DMF |
| | | | solution, 400 mL DMF directly |
| | | | charged into the reactor) |

| | | | |
|---|---|---|---|
| 11 | Resin bound drying temperature | 22° | C. |
| 12 | Resin bound drying vacuum conditions | 1-3 | mbar |
| 13 | Resin bound drying duration | 94 | h |
| 14 | Mass of the dried and discharged resin bound | 3484 | g |
| | intermediate | | |

Molecular weight increase from the initial Sieber resin to the
protected peptide on resin = 5968.44 g/mol

| | | | |
|---|---|---|---|
| 15 | Theoretical weight gain of the protected | 2746 | g |

| | peptide on resin | |
|---|---|---|
| 16 | Actual weight gain | 2836 |
| 17 | Yield of SPPS step | 103.3% |

The detailed coupling sequence and step conditions in the various cycles are listed in Table 4.

TABLE 4
Detailed Conditions for the Individual Cycles in the SPPS Build

| | | | | Number and | | |
|---|---|---|---|---|---|---|
| | | | Number and | Duration of | Coupling | Number and |
| | | | Duration of | Post- | Stir | Duration of |
| AA¹ | Cycle | AA/Building | Deprotection | Deprotection | Time | Post-Coupling |
| # | # | Block | Treatments | DMF Washes | [h] | DMF Washes |
| — | — | Sieber Resin | — | — | 2 (swell) | 4 × 5 min |
| 39 | 1 | Fmoc-L-Ser(O^{t}Bu)—OH | 3 × 10 min | 5 × 5 min | 1.5 | 4 × 5 min |
| 38 | 2 | Fmoc-L-Pro-OH•H₂O | 3 × 10 min | 5 × 5 min | 1.5 | 4 × 5 min |
| 37-36 | 3 | Fmoc-L-Pro-L-Pro-OH | 3 × 10 min | 5 × 5 min | 1.5 | 4 × 5 min |
| 35 | 4 | Fmoc-L-Ala-OH•H₂O | 3 × 10 min | 5 × 5 min | 1.5 | 4 × 5 min |
| 34 | 5 | Fmoc-Gly-OH | 3 × 10 min | 5 × 5 min | 1.5 | 4 × 5 min |
| 33 | 6 | Fmoc-L-Ser(O^{t}Bu)—OH | 3 × 10 min | 5 × 5 min | 1.5 | 4 × 5 min |
| 32 | 7 | Fmoc-L-Ser(O^{t}Bu)—OH | 3 × 10 min | 5 × 5 min | 1.5 | 4 × 5 min |
| 31 | 8 | Fmoc-L-Pro-OH•H₂O | 3 × 10 min | 5 × 5 min | 1.5 | 4 × 5 min |
| 30-29 | 9 | Fmoc-Gly-Gly-OH | 3 × 10 min | 5 × 5 min | 1.5 | 4 × 5 min |
| 28 | 10 | Fmoc-L-Ala-OH•H₂O | 3 × 10 min | 5 × 5 min | 1.5 | 4 × 5 min |
| 27 | 11 | Fmoc-L-Ile-OH | 3 × 10 min | 5 × 5 min | 1.5 | 4 × 5 min |
| 26 | 12 | Fmoc-L-Leu-OH | 3 × 10 min | 5 × 5 min | 1.5 | 4 × 5 min |
| 25 | 13 | Fmoc-L-Trp(Boc)-OH | 3 × 10 min | 5 × 5 min | 1.5 | 4 × 5 min |
| 24 | 14 | Fmoc-L-Gln(Trt)-OH | 3 × 10 min | 5 × 5 min | 1.5 | 4 × 5 min |
| 23 | 15 | Fmoc-L-Val-OH | 3 × 10 min | 5 × 5 min | 1.5 | 4 × 5 min |
| 22 | 16 | Fmoc-L-Phe-OH | 3 × 10 min | 5 × 5 min | 1.5 | 4 × 5 min |
| 21 | 17 | Fmoc-L-Ala-OH•H₂O | 3 × 10 min | 5 × 5 min | 1.5 | 4 × 5 min |
| 20 | 18 | ivDde-L-Lys(Fmoc)-OH | 3 × 10 min | 5 × 5 min | 6 | 4 × 5 min |
| 20.1 | 19 | Fmoc-AEEA-OH | 5 × 10 min | 5 × 5 min | 1.5 | 4 × 5 min |
| 20.2 | 20 | Fmoc-AEEA-OH | 3 × 10 min | 5 × 5 min | 1.5 | 4 × 5 min |
| 20.3 | 21 | Fmoc-L-Glu-O^{t}Bu | 3 × 10 min | 5 × 5 min | 6 | 4 × 5 min |
| — | — | Recoupling of Fmoc-L-Glu-O^{t}Bu | — | — | 6 | 4 × 5 min |
| 20.4 | 22 | 20-(tert-butoxy)-20-oxoicosanoic acid | 3 × 10 min | 5 × 5 min | 22 | 4 × 5 min |
| — | — | Deprotection of ivDde with 3% v/v | 1 × 16 h | 5 × 5 min | — | — |
| | | hydrazine monohydrate/DMF | | | | |
| 19 | 23 | Fmoc-L-Gln(Trt)-OH | — | — | 3 | 4 × 5 min |
| 18 | 24 | Fmoc-L-Ala-OH—H₂O | 3 × 10 min | 5 × 5 min | 1.5 | 4 × 5 min |
| 17 | 25 | Fmoc-L-Ile-OH | 3 × 10 min | 5 × 5 min | 3 | 4 × 5 min |
| 16 | 26 | Fmoc-L-Lys(Boc)-OH | 3 × 10 min | 5 × 5 min | 3 | 4 × 5 min |
| 15 | 27 | Fmoc-L-Asp(OtBu)—OH | 3 × 10 min | 5 × 5 min | 1.5 | 4 × 5 min |
| 14 | 28 | Fmoc-L-Leu-OH | 3 × 10 min | 5 × 5 min | 1.5 | 4 × 5 min |
| 13 | 29 | Fmoc-Aib-OH | 3 × 10 min | 5 × 5 min | 1.5 | 4 × 5 min |
| 12 | 30 | Fmoc-L-Ile-OH | 3 × 10 min | 5 × 5 min | 6 | 4 × 5 min |
| — | — | Recoupling of Fmoc-L-Ile-OH | — | — | 6 | 4 × 5 min |
| 11-10 | 31 | Fmoc-L-Tyr(^{t}Bu)-L- | 3 × 10 min | 5 × 5 min | 3 | 7 × 5 min |
| | | Ser(Ψ(Me,Me)pro)-OH | | | | |
| 9 | 32 | Fmoc-L-Asp(O^{t}Bu)—OH | 3 × 10 min | 5 × 5 min | 1.5 | 4 × 5 min |
| 8 | 33 | Fmoc-L-Ser(O^{t}Bu)—OH | 3 × 10 min | 5 × 5 min | 1.5 | 4 × 5 min |
| 7 | 34 | Fmoc-L-Thr(O^{t}Bu)—OH | 3 × 10 min | 5 × 5 min | 1.5 | 4 × 5 min |
| 6 | 35 | Fmoc-L-Phe-OH | 3 × 10 min | 5 × 5 min | 6 | 4 × 5 min |
| 5-4 | 36 | Fmoc-Gly-L-Thr(Ψ(Me,Me)pro)-OH₃ | 3 × 10 min | 5 × 5 min | 1.5 | 4 × 5 min |
| 3 | 37 | Fmoc-L-Glu(O^{t}Bu)—OH•H₂O | 3 × 10 min | 5 × 5 min | 6 | 4 × 5 min |
| — | — | Recoupling of Fmoc-L- | — | — | 6 | 4 × 5 min |
| | | Glu(O^{t}Bu)—OH•H₂O | | | | |
| 2 | 38 | Fmoc-β-Ala-OH | 3 × 10 min | 5 × 5 min | 6 | 4 × 5 min |
| — | — | Recoupling of Fmoc-β-Ala-OH | — | — | 6 | 4 × 5 min |
| 1 | 39 | 2-(3-cyano-5-fluorophenyl)-2- | 3 × 10 min | 5 × 5 min | 1.5 | 5 × 5 min |
| | | methylpropanoic acid | | | | |

| | | | |
|---|---|---|---|
| — | — | Final washes | 1 × 10 min DMF |
| | | | 4 × 5 min 2-PrOH |
| | | | 4 × 5 min TBME |

¹Amino acid.

### Cleavage/Global Deprotection and Precipitation

The resin-bound material, obtained after the SPPS step, was subjected to cleavage/global deprotection in multiple batches to afford the crude peptide.

TFA (7.2 mL/g of resin bound material) was charged in a jacketed reactor. Deionized water (0.4 mL/g of resin bound material) and TIS (0.4 mL/g of resin bound material) were added, and the mixture was stirred was cooled to 0° C. The resin bound material was charged portion wise into the reactor over 5 minutes. The reaction mixture was then warmed to 20° C. and stirred for 2 hours at this temperature before it was filtered. The resin was washed once with fresh cocktail mixture at ambient temperature (0.8 mL/g of resin bound material).

The filtrate was transferred into a jacketed reactor and cooled to 0° C. Ice-cold TBME (8.8 mL/g of resin bound material) was then added dropwise.

Another portion of ice-cold TBME (44 mL/g of resin bound material) was added in one portion. The resulting suspension was then warmed to 20° C. and maintained at this temperature for 30 minutes. The crude precipitate was filtered, and the filter cake was washed twice with TBME (8.8 mL/g of resin bound material for each wash). The isolated solid was dried at 1-3 mbar and 22° C. for about 16 hours.

The crude peptide was isolated as an off-white powder (2.23 kg in quantitative crude yield).

### Example 6: Purification by HPLC

Screening: The crude peptide was dissolved in 35% w/w AcOH, 5% w/w MeCN, and 60% w/w water. Purification of the peptide solution was screened using the following pre-equilibrated C4, C8, C18, di-C4 and phenyl modified silica resins with pore sizes of 10, 12, 15 or 30 nm and particle sizes of 10 micrometer: Daisopak 100-10-C4-NP, Daisopak 100-10-C4Ph-HP, Daisopak 100-10-C8-PK, Daisopak 100-10-ODS-P, Kromasil 100-10-C4, Kromasil 100-10-diC4, Kromasil 100-10-C8, Kromasil 100-10-C18, Kromasil 100-10-Phenyl, YMC Triart 120-10-C4-S, YMC Triart 120-10-C8-S, YMC Triart 120-10-C18-S, NanoMicro UniSil Revo 150-10-C18, Zeochem Zeosphere 120-10-C18, Zeochem Zeosphere 300-10-C4). The crude peptide solution was applied to the test resin, washed with a low organic solvent buffer, and then eluted using a linear organic solvent gradient. Buffer conditions with acidic (0.1% trifluoroacetic acid, 50 mM ammonium dihydrogen phosphate, or 50 mM sodium dihydrogen phosphate) and basic conditions (50 mM ammonium acetate, 50 mM sodium acetate, or 50 mM ammonium hydrogen carbonate) in a mixture of water and organic solvent (acetonitrile, ethanol, isopropanol) were used. Effluent was collected in fractions and the fractions were analyzed using analytical RP HPLC.

### Two Dimensional Purification

Reversed Phase Purification 1 (RP1): The crude peptide was dissolved to 20 mg/mL in 35% AcOH, 5% MeCN, and 60% water (% w/w). The solution was stirred for at least 18 hours. The crude solution was then filtered (0.45 μm pore size) and loaded onto a pre-equilibrated Kromasil 100-10-C18 packed column. The column was washed with a mixture of A (90% water, 0.1% TFA, and 10% MeCN, % w/w) and B (20% water, 0.1% TFA and 80% MeCN, % w/w) buffers resulting in 32% MeCN for at least 20 minutes and prepared for elution by increasing the mixture concentration of MeCN from 32% to 42%. The peptide was eluted from the column using a 0.04% MeCN increase per minute until elution was complete. The effluent was fractionated and assayed for purity using RP-HPLC. The column was then washed with 76% MeCN for at least 18 minutes. The column was then re-equilibrated using 32% ACN for at least 20 minutes prior to the next injection sequence.

In some cases, fractions that qualified by purity and impurity content were pooled. Fractions that did not meet the purity criteria were combined for recycle injections after all of the primary injections were complete. Upon completion of pooling, the intermediate was assayed for purity and single largest impurity.

Reversed Phase Purification 2 (RP2): To the RP1 solution, two times the volume of 80% water and 20% MeCN (% v/v) was added and loaded onto a pre-equilibrated Kromasil 100-10-C18 packed column. The column was washed with a mixture of A (50 mM ammonium acetate and 10% MeCN, % w/w, pH 8.5) and B (MeCN) buffers resulting in 10% MeCN for at least two minutes and prepared for elution by increasing the mixture concentration of MeCN from 10% to 28%. The peptide was eluted from the column using a 0.19% MeCN increase per minute until elution was complete. The effluent was fractionated and assayed for purity using RP-HPLC. The column was washed with 75% MeCN for at least 14 minutes.

Fractions that qualified by purity and impurity content were pooled. In some cases, fractions that did not meet the purity criteria were combined for recycle injections after all of the primary injections are complete. Upon completion of pooling, the intermediate was assayed for purity and single largest impurity.

### One Dimensional Purification

The crude mixture was dissolved at a concentration of 40 g/L using a solvent mixture of 50% acetonitrile in water. The solution was stirred for 10 minutes and then heated to 40° C. for 90 minutes to facilitate decarboxylation. The solution was cooled to room temperature for 10 minutes and then adjusted to pH 8.0 by adding 5% aqueous ammonium hydroxide. The solution was stirred for 10 minutes to facilitate isoacylisomer conversion and then diluted with water to achieve a final concentration of 20 g/L.

A column was packed with Daisopak 100-10-C8 (4.6×250 mm) stationary phase and was equilibrated with a mixture of A (50 mM ammonium acetate and 5% acetonitrile, % w/w) and B (50 mM ammonium acetate and 70% acetonitrile, % w/w) buffers (70% v/v buffer A/30% v/v buffer B; 24.5% w/w acetonitrile content). The peptide solution was loaded onto the equilibrated column followed by washing with the equilibration mixture for at least ten minutes. The peptide was eluted from the column using a 0.33% MeCN increase per minute until elution was complete. The effluent was fractioned, and the fractions were assayed for purity using analytical RP-HPLC. The column was washed with 66.8% MeCN for at least ten minutes. The column was then re-equilibrated using 24.5% ACN for at least ten minutes before the next injection sequence. Fractions that qualified by purity and impurity content were pooled. In some cases, fractions that did not meet the purity criteria were combined for recycle injections after all of the primary injections were complete. Upon completion of pooling, the intermediate was assayed for purity and single largest impurity.

One Dimensional Purification using Multicolumn Countercurrent Solvent Gradient Purification (MCSGP)

The crude mixture was dissolved at a concentration of 40 g/L using a solvent mixture of 50% acetonitrile in water. The solution was stirred for 16 hours to facilitate decarboxylation. The solution was adjusted to pH 8.0 by adding 5% aqueous ammonium hydroxide and then diluted with water to achieve a final concentration of 20 g/L. The solution was stirred for 4 hours to facilitate isoacylisomer conversion and then diluted with water.

Two columns packed with stationary phase Daisopak 100-10-C8 (4.6×250 mm) were equilibrated with a mixture of A (30 mM ammonium acetate and 5% acetonitrile, % v/v) and B (30 mM ammonium acetate and 70% acetonitrile, % v/v) buffers (70% v/v buffer A/30% v/v buffer B). The peptide solution was loaded onto the first column followed by washing with the equilibration mixture for at least one column volume. The peptide was eluted from the column using a 0.5% buffer B increase per minute. The weakly adsorbing overlap region (UV criterion with time delay, time window before main peak) was loaded onto the second column, the main peak was collected (UV criterion), and the strongly adsorbing overlap region (time window after main peak) was loaded onto the second column using inline dilution to less than 30% buffer B content. Column 1 was washed with 95% buffer B and re-equilibrated with 30% buffer B. Crude peptide solution was loaded into the second column, which already was loaded with the overlap region material from the first elution. The sequence of washing, elution and loading onto the other column was continuously repeated until all crude peptide solution was purified. The combined pool of collected main peak from all elutions was assayed for purity and impurities.

### Desalting Step (Removal of Excess Ions, e.g., NH₄OAc)

Two volumes of 70% water and 30% MeCN (% w/w) were added to the solution generated in RP2 described above, then loaded onto a pre-equilibrated Kromasil 100-10-C18 packed column. The column was washed with a mixture of A (95% water, 5% MeCN, % w/w) and B (MeCN) buffers resulting in 15% MeCN for at least 60 minutes and prepared for elution by increasing the mixture concentration of MeCN from 15% to 60%. The peptide was eluted from the column under isocratic conditions until elution was complete. The effluent was fractionated and assayed for purity using RP-HPLC. The column was re-equilibrated with 15% MeCN for at least 20 minutes prior to the next injection sequence. Upon completion of pooling the fractions that qualify, the intermediate was assayed for purity and single largest impurity purity. The process yields the product with an average pool purity of ≥95.0%.

Desalting and Solvent Change by Tangential Flow Filtration (TFF) (Removal of Excess Ions, e.g. NH₄OAc)

In the first step of the TFF process, the RP2 solution is pre-concentrated by using a membrane with a cut-off showing acceptable rejection of the peptide (e.g., 0.45 kDa, 1 kDa, 2 kDa, 3 kDa, or 5 kDa), manufactured from a suitable membrane material (e.g., cellulose acetate, or ceramic) showing reduced membrane-peptide interactions. The transmembrane pressure (TMP) used within the tangential flow filtration ranges between 2 to 9 bar. A feed flow of up to 6.5 l/min was used based on the membrane geometry. After the targeted concentration is reached, diafiltration using a mixture of 2-propanol/water is used to remove acetonitrile and excess ions. The targeted limits (solvent composition 2-propanol/water 50/50 to 70/30 (w/w), ammonium content below 1.5% (w/w)) of the retentate are reached after 4 to 8 diavolumes; afterwards the product solution is further concentrated until the targeted feed concentration of 10-16% peptide is reached. Finally, the system is rinsed to reduce yield losses.

### Example 7: Isolation of the Compound of Formula (VII) (CT-388)

### Isolation by Spray Drying

For spray drying of the compound of formula (VII), an aqueous organic solvent system was used. Spray drying experiments were carried out on a Büchi S-300 laboratory spray dryer with an inert loop S-395 for closed-loop spray drying and a high-efficiency cyclone for collection of the powder or on a GEA Niro SD Micro spray dryer. Nitrogen gas was used for atomization and as drying gas. Atomization was achieved by using a two-fluid nozzle. The heater started at the defined set point temperature. After the gas temperature stabilized, an aqueous solvent mixture of the same composition as the feed flow was sprayed. The feed rate was adjusted until the targeted outlet temperature was reached. After the outlet temperature stabilized, the feed solution started to spray, and the feed flow was finetuned to achieve the targeted outlet temperature. After spraying the feed solution, pure solvent was fed to end the spray drying process. The powder collected in the cyclone was isolated and analyzed. If the desired limit of residual solvent was not achieved, a secondary drying step was performed using a vacuum oven (using an applied vacuum of 10 mbar and drying temperature of 40° C. overnight).

### Spray Drying of Peptide Solution in Aqueous Ethanol

Spray drying of a 10% (w/w) peptide solution in 70% (w/w) aqueous ethanol was conducted as described above. The process parameters as shown in Table 5 were used.

TABLE 5
Process Parameters of Spray Drying a Solution in Ethanol

| Parameter | Set-point |
|---|---|
| Concentration [% w/w] | 10 |
| Aq. ethanol conc [% w/w] | 70 |
| Outlet temperature (Tout) [° C.] | 60 |
| Inlet temperature (Tin) [° C.] | 120 |

The material was collected as white powder with a yield of 82%. Purity of the collected material was analyzed; no decomposition of product was observed. After secondary drying an EtOH content of <500 ppm for ethanol was reached.

LC-MS (RP-HPLC): RT 19.074 min, m/z 1608.5119 [M+3H]³⁺, translates into a neutral monoisotopic mass of 4822.5139 u (4822.5049 u theoretical).

### Spray Drying of Peptide Solution in Aqueous 2-Propanol

Spray drying of a 10% (w/w) peptide solution in aqueous 2-propanol (10% (w/w) in 70% (w/w) aqueous 2-propanol) was conducted as described under General Procedure above. The process parameters as shown in Table 6A were used.

TABLE 6A
Process Parameters of Spray Drying a Solution in 2-Propanol

| Parameter | Lower Limit | Upper Limit |
|---|---|---|
| Concentration [% w/w] | 2 | 16 |
| Aq. 2-propanol conc [% w/w] | 40 | 70 |
| Outlet temperature (Tout) [° C.] | 60 | 85 |
| Inlet temperature (Tin) [° C.] | 120 | 145 |
| Atomization pressure [bar] | 0.23 | 0.88 |

The material was collected as white powder with a yield of 66% to 83%. Purity of the collected material was analyzed; no decomposition of product was observed.

### Isolation by Spray Drying (Production Scale)

General procedure: Spray drying experiments were carried out on a GEA PSD1 spray drying equipped with a two-fluid nozzle for atomization. Nitrogen was used as atomization and drying gas.

Before batch initiation, the spray dryer was stabilized on the targeted set-points for inlet, outlet temperatures and atomization gas pressure using a solvent mixture matching the composition of the feed solution. Lower and upper limits for the feed compositions and the process parameters used within these trials are shown in Table 6B. Once stable conditions were established, the feed solution was sprayed instead of the stabilization liquid. The powder was collected in the cyclone. After the feed was sprayed, the spray process ended by switching back to the stabilization liquid again.

TABLE 6B
Parameters of Spray Drying a Solution
in Aq. 2-Propanol (Production Scale)

| Parameter | Lower Limit | Upper Limit |
|---|---|---|
| Concentration [% w/w] | 10 | 18 |
| Aq. 2-propanol conc [% w/w] | 40 | 60 |
| Inlet temperature [° C.] | 120 | 130 |
| Outlet temperature [° C.] | 60 | 75 |

The material was collected as white powder with a yield of 96% to 102%. Purity of the collected material was analyzed; no decomposition of product was observed.

### Isolation by Freeze-Drying

The compound of formula (VII) (100 mg) was added to a solvent mixture of H₂O/MeCN 85:15 (v/v, 2 mL) at ambient temperature. To the resulting suspension, aqueous acetic acid was added until the solution became clear (approx. 400 μL). The solution was filtered over a syringe filter (Nylon, 0.45 μm) and then subsequently loaded by a feed pump (feed flow rate 1.9 mL/min) onto a RP-HPLC column (Kromasil 100-10-C18; dimension 4.6×250 mm). The HPLC run was performed using a water/MeCN gradient (Table 7A). The collected fraction (with a retention time of 35-48 minutes) was diluted with water (9 mL), frozen and lyophilized. 85 mg of a colorless solid was obtained.

TABLE 7A
Buffer Gradient Used (Buffer A: Water; Buffer B: MeCN)

| Time [min] | Buffer B [% ] |
|---|---|
| 0 | 15 |
| 60 | 15 |
| 62 | 60 |
| 92 | 60 |
| 94 | 15 |
| 114 | 15 |

### Preparation of the Penta-Sodium Salt of the Compound of Formula (VII) by Freeze-Drying

The compound of formula (VII) (3.01 g) was dissolved in a solvent mixture H₂O/MeCN 50:50 (% v/v, 75 mL) at ambient temperature. To the resulting solution, 1 M aqueous NaOH solution (2.84 mL, target 5 equivalents) was added (amount of sodium hydroxide calculated based on the assay and targeted sodium equivalents). The solution was frozen and lyophilized. 2.39 g of a colorless solid was obtained.

### Example 7.2.3: Preparation of the Acetate Salt of the Peptide of Formula VII by Freeze-Drying

The compound of formula (VII) (203 mg) is dissolved in a solvent mixture of H₂O/MeCN 4:3 (% v/v, 7 mL) at ambient temperature. To the resulting solution, aqueous acetic acid (1%-v/v, 16 mL, target 1 equivalent) was added (amount of sodium hydroxide calculated based on the assay of the peptide and the targeted sodium equivalents). To clear the resulting cloudy solution, MeCN (approx. 6 mL) was added. The solution was frozen and lyophilized. 211 mg of a colorless solid was obtained.

### Preparation of the Ammonium Salt of the Compound of Formula (VII) by Freeze-Drying

The compound of formula (VII) (1.02 g) is dissolved in a solvent mixture of H₂O/MeCN 95:05 (% v/v, 24.9 mL) at ambient temperature. 1 M aqueous NH₄OH solution (1.040 mL, target 5 equivalents) was added to the resulting solution (the amount of ammonium hydroxide is calculated based on the assay and targeted sodium equivalents). The solution is frozen and lyophilized. 0.9 g of a white solid is obtained.

### Isolation by Precipitation

The compound of formula (VII) (12 g) was dissolved in methanol (36 mL) at ambient temperature. Cold TBME was added to the resulting solution under stirring to −10° C. within 10 to 20 minutes. Additionally, the resulting suspension was maintained at −10° C., filtered and washed with cold TBME. Subsequently, the wet material was dried at 45° C. under vacuum. The peptide was isolated as white powder in 95% yield (11.5 g).

### Preparation of the Acetate Salt of the Compound of Formula (VII) by Precipitation

In a first step, the obtained peptide solution after purification (volume approx. 15400 mL) is concentrated to half its initial volume by distillation (pressure 70 mbar, water bath temperature 30° C.). The volume was replenished with neat 2-propanol, and distillation was repeated. This procedure was continued until the residual water content after the last 2-propanol addition is below 5%.

The filtrate (volume 7700 mL) was mixed with water (38 mL) to obtain a solution with 5 volume % water. The distillation vessel was rinsed in two portions with a solvent mixture of 2-propanol: water 95:5 (% v/v, 800 mL).

17200 mL of a solvent mixture MTBE/n-heptane 1:1 (% v/v) as cooled to between-10 to −15° C. within approximately two hours. Under stirring, the clear peptide solution (8538 mL) was added over a period of 45 minutes. The milky suspension was stirred at −10° C. overnight. Afterwards, the suspension was filtered at −10° C. The filter cake was washed with ice-cold MTBE/n-heptane 1:1 (% v/v).

Drying the wet material was initiated by displacing the residual organic solvents using a MeCN-saturated nitrogen stream. Afterwards, drying as completed in a second step by using a water saturated nitrogen stream. 132.4 g of a colorless solid was obtained. LC-MS (RP-HPLC): RT 18.918 min, m/z 1608.5102 [M+3H]³⁺, translates into a neutral monoisotopic mass of 4822.5088 u (4822.5049 u theoretical).

### Preparation of the HCl Salt of the Compound of Formula (VII) by Freeze-Drying

Reversed Phase Purification 1 (RP1): The crude peptide was dissolved to 30 mg/mL in 10% AcOH, 40% MeCN, 50% water (% v/v) and filtered (0.45 μm). To the crude solution was added an equal volume of A (water, 0.1% TFA) buffer, diluted three times with purified water and loaded onto a pre-equilibrated Luna-C18 packed column. The column was washed with a mixture of A (water, 0.1% TFA, % v/v) and B (20% water, 0.09% TFA and 80% MeCN, % v/v) buffers resulting in 8% MeCN for at least six min. Subsequent equilibration of the column is performed at 40% MeCN for at least six minutes. The peptide is eluted from the column using a 0.66% MeCN increase per minute until elution was complete. The effluent was fractionated and assayed for purity using RP-HPLC. The column was washed with 80% MeCN for at least six minutes.

Fractions that qualify for inclusion were pooled. In some cases, fractions that did not meet the purity criteria were combined for recycle injections after all of the primary injections were complete. Recycle injections were processed and pooled using the primary injection criteria; however, only main peak fractions were forward processed and no further recycling was performed. Upon completion of pooling the fractions that qualify, the intermediate was assayed for purity and single impurity.

Ion Exchange Chromatography (IEX): To the example RP1 solution was added an equal volume of A (water, 0.05% HCl, % v/v) buffer, diluted three times with purified water and loaded onto a pre-equilibrated Luna-C18 packed column. Subsequent equilibration of the column was performed with A (water, 0.05% HCl, % v/v) and B (20% water, 0.05% HCl, 80% MeCN, % v/v) buffer at 4% MeCN for at least 20 min followed by another equilibration at 36% MeCN for at least eight minutes. The peptide was eluted from the column using a 0.73% MeCN increase per minute until elution was complete. The effluent was fractionated and assayed for purity using RP-HPLC. The column was washed with 80% MeCN for at least six minutes.

Fractions that qualify for inclusion were pooled. In some cases, fractions that did not meet the purity criteria may be combined for recycle injections after all of the primary injections are complete. Recycle injections were processed and pooled using the primary injection criteria; however, only main peak fractions were forward processed and no further recycling was performed. Upon completion of mainstream pooling, the intermediate was assayed for purity and single impurity. The process yields the product with an average pool purity of ≥95.0%.

The final pool after purification was diluted with an equal volume of water and filtered (0.2 μm). The resulting solution was frozen and lyophilized. The peptide was isolated in 22% yield, calculated from the starting crude peptide solution. LC-MS (RP-HPLC): RT 18.406 min, m/z 1608.5090 [M+3H]³⁺, translates into a neutral monoisotopic mass of 4822.5052 u (4822.5049 u theoretical).

### Preparation of Various Salts of the Compound of Formula (VII) by Freeze-Drying

Salts with cationic counter-ion: The peptide (200 mg) was dissolved in a solvent mixture of H₂O/MeCN 50:50 (v/v, 7 mL) at ambient temperature. To the resulting solution, the calculated amount of the counter-ion (with a 1:5 equiv. target ratio of peptide: counter-ion) as aqueous solution was added. If the resulting mixture was cloudy, MeCN was added until the solution became clear. The solution was then frozen and lyophilized. The salt was obtained as a colorless solid. The preparation of the salts is described in Table 7B.

TABLE 7B
Salts with Cationic Counter-Ion

| Example # | Counter-ion | Added as | Yield |
|---|---|---|---|
| a | Ammonium | Aqueous ammonium hydroxide | 170 mg |
| | | solution | |
| b | Potassium | Aqueous potassium hydroxide | 240 mg |
| | | solution | |
| c | Sodium | Aqueous sodium hydroxide | 180 mg |
| | | solution | |

Salts with anionic counter-ion: The peptide (200 mg) was dissolved in a solvent mixture of H₂O/MeCN 50:50 (v/v, 7 mL) at ambient temperature. To the resulting solution the calculated amount of the counter-ion (with a 1:1 equiv. target ratio of peptide: counter-ion) as aqueous solution was added. In case the resulting mixture was cloudy, MeCN was added until the solution became clear. The solution then was frozen and lyophilized. The salt was obtained as a colorless solid. The preparation of the salts is described in Table 7C.

TABLE 7C
Salts with Anionic Counter-Ion

| Example # | Counter-ion | Added as | Yield |
|---|---|---|---|
| d | Acetate | Aqueous acetic acid solution | 207 mg |
| e | Chloride | Aqueous hydrochloride solution | 180 mg |
| f | Phosphate | Aqueous phosphoric acid solution | 230 mg |

### Example 8: Solid-Phase Synthesis of the Compound of Formula (VII) Without Using Pseudoproline Residues

### Reaction Scheme and Peptide Assembly

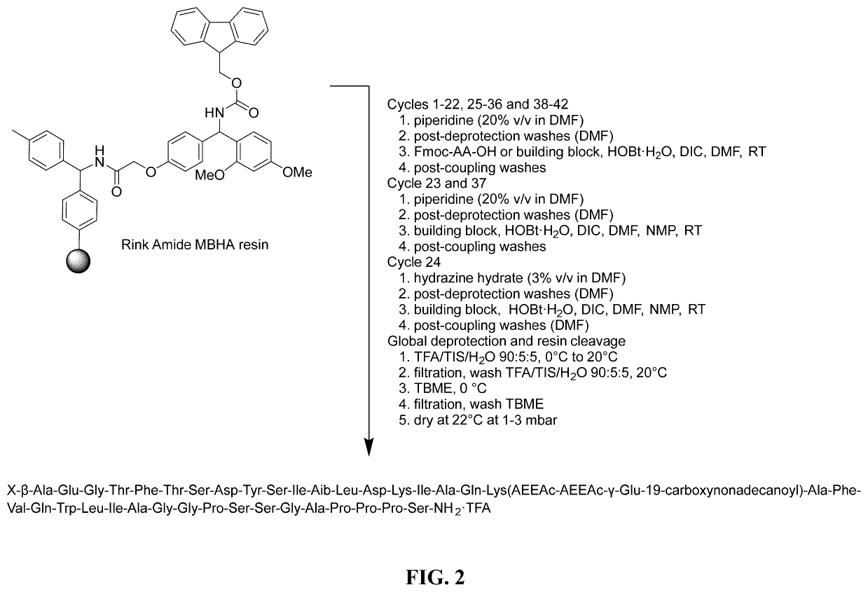

The linear peptide including the side chain was synthesized on a Protein Technologies Tribute automated peptide synthesizer on a scale of 0.75 mmol (Table 8, FIG. 2). Rink Amide MBHA resin (200-400 mesh, 0.67 mmol/g, 1% DVB) was used.

TABLE 8
Building Blocks Coupled in Sold-Phase Peptide Synthesis
Amino acid / building block coupled (SEQ ID NO: 1)

| 1 | 2 | 3 | 4 | 5 | 6 |
|---|---|---|---|---|---|
| Ser³⁹ | Pro³⁸ | Pro³⁷ | Pro³⁶ | Ala³⁵ | Gly³⁴ |
| 7 | 8 | 9 | 10 | 11 | 12 |
| Ser³³ | Ser³² | Pro³¹ | Gly³⁰- | Ala²⁸ | Ile²⁷ |
| | | | Gly²⁹ | | |
| 13 | 14 | 15 | 16 | 17 | 18 |
| Leu²⁶ | Trp²⁵ | Gln²⁴ | Val²³ | Phe²² | Ala²¹ |
| 19 | 20 | 21 | 22 | 23 | 24 |
| Lys²⁰ | AEEA^{20.1} | AEEA^{20.2} | gGlu^{20.3} | C₂₀ | Gln¹⁹ |
| | | | | acid^{20.4} | |
| 25 | 26 | 27 | 28 | 29 | 30 |
| Ala¹⁸ | Ile¹⁷ | Lys¹⁶ | Asp¹⁵ | Leu¹⁴ | Aib¹³ |
| 31 | 32 | 33 | 34 | 35 | 36 |
| Ile¹² | Ser¹¹ | Tyr¹⁰ | Asp⁹ | Ser⁸ | Thr⁷ |
| 37 | 38 | 39 | 40 | 41 | 42 |
| Phe⁶ | Thr⁵ | Gly⁴ | Glu³ | β-Ala² | X¹ = 2-(3-cyano-5- |
| | | | | | fluorophenyl)-2- |
| | | | | | methylpropanoic |
| | | | | | acid |

The amounts and concentrations of the reagents and solvents used for this build are listed in Table 9.

TABLE 9
Conditions Used for the SPPS Build On

| Entry | Condition/Parameter | | |
|---|---|---|---|
| 1 | Initial Rink Amide MBHA resin loading | 0.67 | mmol/g |
| 2 | Amount DMF for each post-coupling wash | 11 | mL/g resin |
| 3 | Amount piperidine in DMF (20% v/v) for each | 11 | mL/g resin |
| | Fmoc deprotection treatment | | |
| 4 | Amount N₂H₄•H₂O in DMF (3% v/v) for each | 11 | mL/g resin |
| | ivDde deprotection treatment | | |
| 5 | Amount DMF for each post-deprotection wash | 11 | mL/g resin |
| 6 | Amount 2-PrOH for each de-swelling wash | 10 | mL/g resin |
| 7 | Amount TBME for each de-swelling wash | 10 | mL/g resin |
| 8 | Pre-activation time for all couplings | 2 | min |

| | | | |
|---|---|---|---|
| 9 | Reagents used for coupling | 1-22 | 3 equiv Fmoc-AA-OH or |
| | in cycles | | building block (0.25M in |
| | | | DMF) |
| | | | 3 equiv HOBt•H₂O |
| | | | 3 equiv DIC |
| | | 23 | 3 equiv building block (0.25M |
| | | | in NMP and DMF) |
| | | | 3 equiv HOBt•H₂O |
| | | | 3 equiv DIC |
| | | 24, 37 | 6 equiv Fmoc-AA-OH |
| | | | (0.375M in NMP and DMF) |
| | | | 6 equiv HOBt•H₂O |
| | | | 6 equiv DIC |
| | | 25-36, | 6 equiv Fmoc-AA-OH |
| | | 38-41 | (0.375M in DMF) |
| | | | 6 equiv HOBt•H₂O |
| | | | 6 equiv DIC |
| | | 42 | 6 equiv building block (0.25M |
| | | | in DMF) |
| | | | 2 equiv HOBt•H₂O |
| | | | 2 equiv DIC |

| | | | |
|---|---|---|---|
| 10 | Mass of the dried and discharged resin bound | 5.60 | g |
| | intermediate | | |

Molecular weight increase from the initial Sieber resin to the
protected peptide on resin = 5968.44 g/mol

| | | | |
|---|---|---|---|
| 11 | Theoretical weight gain of the protected | 4.48 | g |
| | peptide on resin | | |
| 12 | Actual weight gain | 3.45 | g |

| | | |
|---|---|---|
| 13 | Yield of SPPS step | 77% |

The detailed coupling sequence and step conditions in the various cycles are listed in Table 10.

TABLE 10
Detailed Conditions for the Individual Cycles in the SPPS Build

| | | | | Number and | | |
|---|---|---|---|---|---|---|
| | | | Number and | Duration of | Coupling | Number and |
| | | | Duration of | Post- | Stir | Duration of |
| AA | Cycle | AA/Building | Deprotection | Deprotection | Time | Post-Coupling |
| # | # | Block | Treatments | DMF Washes | [h] | DMF Washes |
| 39 | 1 | Fmoc-L-Ser(O^{t}Bu)—OH | 1 × 5 min, | 8 × 5 min | 1.5 | 4 × 5 min |
| | | | 1 × 15 min | | | |
| 38 | 2 | Fmoc-L-Pro-OH | 1 × 5 min, | 8 × 5 min | 1.5 | 4 × 5 min |
| | | | 1 × 15 min | | | |
| 37 | 3 | Fmoc-L-Pro-OH | 1 × 5 min, | 8 × 5 min | 1.5 | 4 × 5 min |
| | | | 1 × 15 min | | | |
| 36 | 4 | Fmoc-L-Pro-OH | 1 × 5 min, | 8 × 5 min | 1.5 | 4 × 5 min |
| | | | 1 × 15 min | | | |
| 35 | 5 | Fmoc-L-Ala-OH | 1 × 5 min, | 8 × 5 min | 1.5 | 4 × 5 min |
| | | | 1 × 15 min | | | |
| 34 | 6 | Fmoc-Gly-OH | 1 × 5 min, | 8 × 5 min | 1.5 | 4 × 5 min |
| | | | 1 × 15 min | | | |
| 33 | 7 | Fmoc-L-Ser(O^{t}Bu)—OH | 1 × 5 min, | 8 × 5 min | 1.5 | 4 × 5 min |
| | | | 1 × 15 min | | | |
| 32 | 8 | Fmoc-L-Ser(O^{t}Bu)—OH | 1 × 5 min, | 8 × 5 min | 1.5 | 4 × 5 min |
| | | | 1 × 15 min | | | |
| 31 | 9 | Fmoc-L-Pro-OH | 1 × 5 min, | 8 × 5 min | 1.5 | 4 × 5 min |
| | | | 1 × 15 min | | | |
| 30-29 | 10 | Fmoc-Gly-Gly-OH | 1 × 5 min, | 8 × 5 min | 1.5 | 4 × 5 min |
| | | | 1 × 15 min | | | |
| 28 | 11 | Fmoc-L-Ala-OH | 1 × 5 min, | 8 × 5 min | 1.5 | 5 × 5 min |
| | | | 1 × 15 min | | | |
| 27 | 12 | Fmoc-L-Ile-OH | 1 × 5 min, | 9 × 5 min | 1.5 | 5 × 5 min |
| | | | 1 × 15 min | | | |
| 26 | 13 | Fmoc-L-Leu-OH | 1 × 5 min, | 9 × 5 min | 1.5 | 5 × 5 min |
| | | | 1 × 15 min | | | |
| 25 | 14 | Fmoc-L-Trp(Boc)-OH | 1 × 5 min, | 9 × 5 min | 1.5 | 5 × 5 min |
| | | | 1 × 15 min | | | |
| 24 | 15 | Fmoc-L-Gln(Trt)-OH | 1 × 5 min, | 9 × 5 min | 1.5 | 5 × 5 min |
| | | | 1 × 15 min | | | |
| 23 | 16 | Fmoc-L-Val-OH | 1 × 5 min, | 9 × 5 min | 1.5 | 5 × 5 min |
| | | | 1 × 15 min | | | |
| 22 | 17 | Fmoc-L-Phe-OH | 1 × 5 min, | 9 × 5 min | 1.5 | 5 × 5 min |
| | | | 1 × 15 min | | | |
| 21 | 18 | Fmoc-L-Ala-OH | 1 × 5 min, | 9 × 5 min | 1.5 | 5 × 5 min |
| | | | 1 × 15 min | | | |
| 20 | 19 | ivDde-L-Lys(Fmoc)-OH | 1 × 5 min, | 9 × 5 min | 1.5 | 5 × 5 min |
| | | | 1 × 15 min | | | |
| 20.1 | 20 | Fmoc-AEEA-OH | 1 × 5 min, | 9 × 5 min | 1.5 | 5 × 5 min |
| | | | 1 × 15 min | | | |
| 20.2 | 21 | Fmoc-AEEA-OH | 1 × 5 min, | 9 × 5 min | 1.5 | 5 × 5 min |
| | | | 1 × 15 min | | | |
| 20.3 | 22 | Fmoc-L-Glu-OtBu | 1 × 5 min, | 9 × 5 min | 6 | 5 × 5 min |
| | | | 1 × 15 min | | | |
| 20.4 | 23 | 20-(tert-butoxy)-20- | 1 × 5 min, | 9 × 5 min | 6 | 5 × 5 min |
| | | oxoicosanoic acid | 1 × 15 min | | | |
| — | — | Deprotection of ivDde with 3% | 2 × 1 h, | 9 × 5 min | — | — |
| | | v/v hydrazine monohydrate/DMF | 1 × 4 h, | | | |
| | | | 2 × 1 h | | | |
| 19 | 24 | Fmoc-L-Gln(Trt)-OH | — | — | 1.5 | 5 × 5 min |
| 18 | 25 | Fmoc-L-Ala-OH | 1 × 5 min, | 9 × 5 min | 1.5 | 5 × 5 min |
| | | | 1 × 15 min | | | |
| 17 | 26 | Fmoc-L-Ile-OH | 1 × 5 min, | 9 × 5 min | 1.5 | 5 × 5 min |
| | | | 1 × 15 min | | | |
| 16 | 27 | Fmoc-L-Lys(Boc)-OH | 1 × 5 min, | 9 × 5 min | 1.5 | 5 × 5 min |
| | | | 1 × 15 min | | | |
| 15 | 28 | Fmoc-L-Asp(O^{t}Bu)—OH | 1 × 5 min, | 9 × 5 min | 1.5 | 5 × 5 min |
| | | | 1 × 15 min | | | |
| 14 | 29 | Fmoc-L-Leu-OH | 1 × 5 min, | 9 × 5 min | 1.5 | 5 × 5 min |
| | | | 1 × 15 min | | | |
| 13 | 30 | Fmoc-Aib-OH | 1 × 5 min, | 9 × 5 min | 6 | 5 × 5 min |
| | | | 1 × 15 min | | | |
| 12 | 31 | Fmoc-L-Ile-OH | 1 × 5 min, | 9 × 5 min | 19 | 5 × 5 min |
| | | | 1 × 15 min | | | |
| — | — | Recoupling of Fmoc-L-Ile-OH | — | — | 1.5 | 5 × 5 min |
| 11 | 32 | Fmoc-L-Ser(O^{t}Bu)—OH | 1 × 5 min, | 9 × 5 min | 1.5 | 5 × 5 min |
| | | | 1 × 15 min | | | |
| 10 | 33 | Fmoc-L-Tyr(O^{t}Bu)—OH | 1 × 5 min, | 9 × 5 min | 1.5 | 5 × 5 min |
| | | | 1 × 15 min | | | |
| 9 | 34 | Fmoc-L-Asp(O^{t}Bu)—OH | 1 × 5 min, | 9 × 5 min | 1.5 | 5 × 5 min |
| | | | 1 × 15 min | | | |
| 8 | 35 | Fmoc-L-Ser(O^{t}Bu)—OH | 1 × 5 min, | 9 × 5 min | 1.5 | 5 × 5 min |
| | | | 1 × 15 min | | | |
| 7 | 36 | Fmoc-L-Thr(O^{t}Bu)—OH | 1 × 5 min, | 9 × 5 min | 1.5 | 5 × 5 min |
| | | | 1 × 15 min | | | |
| 6 | 37 | Fmoc-L-Phe-OH | 1 × 5 min, | 9 × 5 min | 1.5 | 5 × 5 min |
| | | | 1 × 15 min | | | |
| 5 | 38 | Fmoc-L-Thr(O^{t}Bu)—OH | 1 × 5 min, | 9 × 5 min | 1.5 | 5 × 5 min |
| | | | 1 × 15 min | | | |
| 4 | 39 | Fmoc-L-Gly-OH | 1 × 5 min, | 9 × 5 min | 1.5 | 5 × 5 min |
| | | | 1 × 15 min | | | |
| 3 | 40 | Fmoc-L-Glu(O^{t}Bu)—OH | 1 × 5 min, | 9 × 5 min | 1.5 | 5 × 5 min |
| | | | 1 × 15 min | | | |
| 2 | 41 | Fmoc-β-Ala-OH | 1 × 5 min, | 9 × 5 min | 1.5 | 5 × 5 min |
| | | | 1 × 15 min | | | |
| 1 | 42 | 2-(3-cyano-5-fluorophenyl)-2- | 1 × 5 min, | 9 × 5 min | 1.5 | 9 × 5 min |
| | | methylpropanoic acid | 1 × 15 min | | | |
| — | — | Final washes | 1 × 10 min DMF | | | |
| | | | 4 × 5 min 2-PrOH | | | |
| | | | 4 × 5 min TBME | | | |

### Synthetic Procedure: Cleavage/Global Deprotection and Precipitation

The resin-bound material, obtained after the SPPS step, was subjected to cleavage/global deprotection in multiple batches to afford the crude peptide.

DTT (0.5 g/g of resin bound material) was charged in a jacketed reactor followed by TFA (7.8 mL/g of resin bound material). The mixture was stirred until all the solids were dissolved (2 minutes). Subsequently, the mixture was cooled to 5° C., and deionized water (0.24 mL/g of resin bound material) and TIS (0.24 mL/g of resin bound material) were added. The addition line was rinsed with a small amount of TFA (0.8 mL/g of resin bound material), and the mixture was stirred and cooled to 2° C. The resin bound material (250 g) was charged into a separate reactor and cooled to −5° C. The pre-cooled cleavage cocktail was then transferred over 5 min to the reactor containing the resin bound material. Subsequently, the reactor agitation started, and the mixture was allowed to warm up to 20° C. in 2° C. increments every 15 min. Upon reaching a reaction temperature of 20° C., the mixture was stirred for 55 min before it was filtered.

The resin was washed once with TFA at ambient temperature (1.1 mL/g of resin bound material). The filtrate was transferred into a jacketed reactor and cooled to −7° C. Ice-cold TBME (53 mL/g of resin bound material) was then added over about four hours while maintaining the internal temperature below 5° C. The resulting suspension was then warmed to 20° C. and maintained at this temperature for 32 minutes. The crude precipitate was filtered, and the filter cake was washed twice with TBME (8.6 mL/g of resin bound material for each wash). The isolated solid was dried at 1-3 mbar and 22° C. for about 24 hours. The crude peptide was isolated as an off-white powder (158 g in 96% crude yield, 81.1% purity, 57.9% assay).

### Purification and Isolation

The crude peptide was purified in analogy to the method described in Example 6 by preparative reversed-phase HPLC and isolated in analogy to the methods described in Example 7.

1. A process of preparing 2-(3-cyano-5-fluorophenyl)-2-methylpropanoic acid (formula (I)): 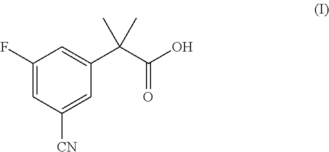
comprising
a) reacting 3-bromo-5-fluorobenzonitrile (formula (II)): 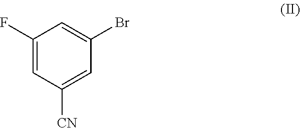
with an isobutyrate source in the presence of a palladium cross coupling catalyst to afford an ester of formula (III) 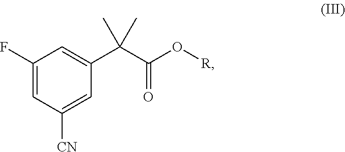
wherein R is C₁₋₄-alkyl, and
b) hydrolyzing the ester of formula (III) to afford 2-(3-cyano-5-fluorophenyl)-2-methylpropanoic acid (formula (I)).

2. The process of claim 1, wherein the palladium cross coupling catalyst is a Pd-trialkylphosphine complex catalyst.

3. The process of claim 2, wherein step a) comprises generating the palladium cross coupling catalyst in situ from a Pd precursor and a trialkylphosphine.

4. The process of claim 1, wherein the isobutyrate source is a C₁₋₄-alkyl isobutyrate or a silyl ketene acetal.

5. The process of claim 1, wherein the isobutyrate source is methyl isobutyrate or methyl trimethylsilyl dimethylketene acetal.

6. The process of claim 1, wherein the isobutyrate source is methyl isobutyrate and the reacting takes place in the presence of a strong base.

7. The process of claim 6, wherein the strong base is a lithium dialkylamide, and wherein the process comprises preparing the lithium dialkylamide in situ from an alkyl lithium compound and a dialkylamine.

8. The process of claim 6, wherein the reacting in the presence of a strong base takes place in a non-polar organic solvent at a reaction temperature between −10° C. and 30° C.

9. The process of claim 4, wherein the reacting of the palladium cross coupling catalyst with the silyl ketene acetal takes place in the presence of a Zn compound as co-activator.

10. The process of claim 9, wherein the silyl ketene acetal is an alkyl trialkylsilyl dialkylketene acetal.

11. The process of claim 9, wherein step a) takes place in a polar organic solvent at a reaction temperature between 40° C. and 100° C.

12. The process of claim 1, wherein step b) comprises hydrolyzing the ester of formula (III) with an alkali hydroxide.

13. The process of claim 12, wherein the alkali hydroxide is lithium hydroxide.

14. The process of claim 12, wherein step b) takes place in the presence of a non-polar organic solvent at a reaction temperature between 0° C. and 100° C.

15. The process of claim 1, wherein step b) is carried out with lithium iodide.

16. 2-(3-cyano-5-fluorophenyl)-2-methylpropanoic acid (formula (I)) produced according to the process of claim 1 for use in a method of preparing a compound of formula (VII), or a pharmaceutically acceptable salt thereof:
(VII)
[SEQ ID NO: 1]
X-β-Ala²-Glu³-Gly⁴-Thr⁵-Phe⁶-Thr⁷-Ser⁸-Asp⁹-
Tyr¹⁰-Ser¹¹-Ile¹²-Aib¹³-Leu¹⁴-Asp¹⁵-Lys¹⁶-Ile¹⁷-
Ala¹⁸-Gln¹⁹-Lys²⁰(AEEAc-AEEAc-γ-Glu-19-
carboxynonadecanoyl)-Ala²¹-Phe²²-Val²³-Gln²⁴-
Trp²⁵-Leu²⁶-Ile²⁷-Ala²⁸-Gly²⁹-Gly³⁰-Pro³¹-Ser³²-
Ser³³-Gly³⁴-Ala³⁵-Pro³⁶-Pro³⁷-Pro³⁸-Ser³⁹-NH₂,
wherein X is 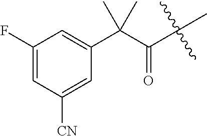 [SMILES: *C(=O)C(C)(C)c1cc(F)cc(C#N)c1]
and
AEEAc is 2-(2-(2-aminoethoxy) ethoxy) acetic acid.

17. A process for preparing a compound of formula (VII), or a pharmaceutically acceptable salt thereof:
(VII)
[SEQ ID NO: 1]
X-β-Ala²-Glu³-Gly⁴-Thr⁵-Phe⁶-Thr⁷-Ser⁸-Asp⁹-Tyr¹⁰-
Ser¹¹-Ile¹²-Aib¹³-Leu¹⁴-Asp¹⁵-Lys¹⁶-Ile¹⁷-Ala¹⁸-Gln¹⁹-
Lys²⁰(AEEAc-AEEAc-γ-Glu-19-carboxynonadecanoyl)-
Ala²¹-Phe²²-Val²³-Gln²⁴-Trp²⁵-Leu²⁶-Ile²⁷-Ala²⁸-Gly²⁹-
Gly³⁰-Pro³¹-Ser³²-Ser³³-Gly³⁴-Ala³⁵-Pro³⁶-Pro³⁷-Pro³⁸-
Ser³⁹-NH₂,
wherein X is 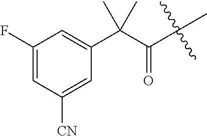 [SMILES: *C(=O)C(C)(C)c1cc(F)cc(C#N)c1]
and
AEEAc is 2-(2-(2-aminoethoxy) ethoxy) acetic acid,
comprising:
a) reacting 2-(3-cyano-5-fluorophenyl)-2-methylpropanoic acid (formula (I)) with the resin-bound peptide: 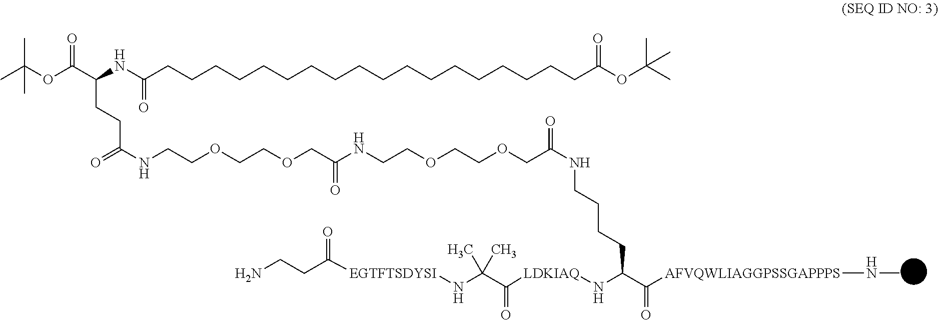
under amide bond-forming conditions.

18. The process of claim 17, further comprising
b) globally deprotecting and cleaving the compound of formula (VII) from the resin using an acid,
c) precipitating the compound of formula (VII) or salt thereof,
d) purifying the compound of formula (VII) or salt thereof, and
e) isolating the compound of formula (VII) or salt thereof.

19-22. (canceled)

23. The process of claim 17, wherein the solid phase synthesis comprises sequentially coupling each of the following to the resin or peptide intermediate bound thereto:
(1) Fmoc-L-Ser(O′Bu)-OH,
(2) Fmoc-L-Pro-OH,
(3) Fmoc-L-Pro-OH,
(4) Fmoc-L-Pro-OH,
(5) Fmoc-L-Ala-OH,
(6) Fmoc-Gly-OH,
(7) Fmoc-L-Ser(O′Bu)-OH,
(8) Fmoc-L-Ser(O′Bu)-OH,
(9) Fmoc-L-Pro-OH,
(10) Fmoc-Gly-Gly-OH,
(11) Fmoc-L-Ala-OH,
(12) Fmoc-L-Ile-OH,
(13) Fmoc-L-Leu-OH,
(14) Fmoc-L-Trp (Boc)-OH,
(15) Fmoc-L-Gln(Trt)-OH,
(16) Fmoc-L-Val-OH,
(17) Fmoc-L-Phe-OH,
(18) Fmoc-L-Ala-OH,
(19) ivDde-L-Lys(Fmoc)-OH,
(20) Fmoc-AEEA-OH,
(21) Fmoc-AEEA-OH,
(22) Fmoc-L-Glu-O′Bu,
(23) 20-(tert-butoxy)-20-oxoicosanoic acid, followed by deprotection of ivDde with 3% v/v hydrazine monohydrate/DMF,
(24) Fmoc-L-Gln(Trt)-OH,
(25) Fmoc-L-Ala-OH,
(26) Fmoc-L-Ile-OH,
(27) Fmoc-L-Lys(Boc)-OH,
(28) Fmoc-L-Asp(O′Bu)-OH,
(29) Fmoc-L-Leu-OH,
(30) Fmoc-Aib-OH,
(31) Fmoc-L-Ile-OH,
(32) Fmoc-L-Ser(O′Bu)-OH,
(33) Fmoc-L-Tyr(O′Bu)-OH,
(34) Fmoc-L-Asp(O′Bu)-OH,
(35) Fmoc-L-Ser(O′Bu)-OH,
(36) Fmoc-L-Thr(O′Bu)-OH,
(37) Fmoc-L-Phe-OH,
(38) Fmoc-L-Thr(O′Bu)-OH,
(39) Fmoc-L-Gly-OH,
(40) Fmoc-L-Glu (O′Bu)-OH,
(41) Fmoc-β-Ala-OH, and
(42) 2-(3-cyano-5-fluorophenyl)-2-methylpropanoic acid.

24-33. (canceled)

34. The process of claim 17, wherein the pharmaceutically acceptable salt of the compound of formula (VII) is an ammonium, potassium, sodium, acetate, chloride, or phosphate salt.
